(19) United States
(12) Patent Application Publication
(10) Pub. No.: US 2023/0218604 A1
(43) Pub. Date: Jul. 13, 2023

(54) PPARy AGONIST FOR TREATMENT OF PROGRESSIVE SUPRANUCLEAR PALSY

(71) Applicant: Coherus Biosciences, Inc., Redwood City, CA (US)

(72) Inventors: Barbara Finck, San Francisco, CA (US); David E. Weinstein, Dobbs Ferry, NY (US); Sarita K. Jain, Sunnyvale, CA (US)

(21) Appl. No.: 17/571,352

(22) Filed: Jan. 7, 2022

(51) Int. Cl. A61K 31/47; A61P 25/28; G01N 33/68

(52) U.S. Cl. CPC A61K 31/47; A61P 25/28; G01N 33/68

(57) ABSTRACT

Methods of treatment of progressive supranuclear palsy or its symptoms, with PPARγ agonists, and in particular, the compound of formula (I) known as INT131:

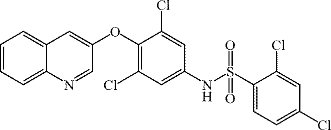 [SMILES: O=S(=O)(Nc1cc(Cl)c(Oc2cnc3ccccc3c2)c(Cl)c1)c1ccc(Cl)cc1Cl]

. Also provided are methods of treating a subject that include selecting a subject having an elevated level of neurofilament light chain protein in a sample obtained from the subject, as compared to a reference level of neurofilament light chain protein, and administering a pharmaceutical composition including a therapeutically effective amount of a compound of formula (I) to the selected subject.

### CROSS-REFERENCE TO RELATED APPLICATIONS

This application is a Continuation of U.S. Application Serial No. 16/497,523, filed Sep. 25, 2019, which is a National Stage application under 35 U.S.C. § 371 of International Patent Application Serial No. PCT/US2018/025923, filed Apr. 3, 2018, which claims priority to U.S. Provisional Pat. Application Serial No. 62/480,838, filed Apr. 3, 2017, and U.S. Provisional Patent Application Serial No. 62/651,653, filed Apr. 2, 2018; the entire contents of all of which are herein incorporated by reference.

### FIELD OF THE INVENTION

The present invention relates to methods of treatment of progressive supranuclear palsy. The present invention also relates, at least in part, to methods using CHS-131 for the treatment of subjects having an elevated level of neurofilament light chain protein in a sample comprising cerebrospinal fluid, blood, serum, or plasma that has been obtained from the subject (e.g., as compared to a reference level of neurofilament light chain protein).

### BACKGROUND OF THE INVENTION

Neurological disorders can cause permanent and irreversible damage that can affect a patient’s quality of life, such as speech, cognitive skills, motor skills, and metabolism. Treatments that would delay the onset of symptoms of a neurological disorder in a subject are desired for patients that are diagnosed as being in the early or middle stages of a neurological disorder.

Progressive Supranuclear Palsy (PSP), a relentless and fatal tauopathy, accounts for ~10% of all parkinsonian cases in the U.S. Patients with PSP present with a variety of motor dysfunctions, including postural instability, falls, gait abnormalities, bradykinesia, vertical gaze palsy, pseudobulbar palsy, and axial rigidity without limb rigidity, as well as cognitive impairments including apathy, loss of executive function, and diminished fluency. The neuropathology of PSP is characterized by an accumulation of abnormal intracellular microtubule-associated protein tau, resulting in insoluble paired helical filaments. The neuropathological presentation of PSP neurodegeneration is localized to the subcortical regions including substantia nigra, globus palladus, and the subthalamic nucleus. The neurodegeneration of PSP is characterized by tissue destruction and cytokine profiles of the activated microglia and astrocytes. These findings are consistent with these activated glial cells as the local sources inflammation and oxidative stress in PSP.

There are approximately 1,400 new diagnosis of PSP per year in the US, with a mean survival 5.9-6.9 years. There are currently no disease modifying treatments for PSP. The current standard of care is palliative. Patients in the advanced stages of the disease often have feeding tubes inserted to avoid choking risks and to provide nutrition.

While there are therapies available to lessen some symptoms of PSP, none protect the brain or slow the deterioration of nerve cells. Current medications to treat symptoms of PSP include dopamine agonists, tricyclic antidepressants, methysergide, Onabotulinumtoxin A (to treat muscle rigidity in the face). However, as the disease progresses, and symptoms worsen, medications may fail to adequately lessen the symptoms.

Accordingly, there is need for new safe and effective treatments of progressive supranuclear palsy.

### SUMMARY OF THE INVENTION

It has now been discovered that the peroxisome proliferator-activated receptor gamma (PPARy) agonist INT131 (CHS131) is effective for treating progressive supranuclear palsy (“PSP”). The PPARγ is a transcription factor belonging to the superfamily of ligand-activated transcription factors, including the steroid, thyroid, and retinoid receptors [Thiemermann, 2004 #12090]. To date, PPARγ agonists have been therapeutic agents for disorders of metabolism, including obesity, Type 2 diabetes mellitus, and dyslipidemia. Recently, the novel, selective PPARy agonist, CHS-131 has been shown to decrease inflammation and to be neuroprotective in patients with relapsing remitting Multiple Sclerosis.

In one aspect, the present invention provides methods of treating progressive supranuclear palsy and symptoms thereof. The methods typically involve administering to a subject in need thereof a therapeutically effective amount of compound INT131 described in U.S. Pat. No. 7,601,841. INT131 is unique among PPARy agonists in that it exerts potent anti-inflammatory effects in the central nervous system without evidence of systemic immunosuppression and is a selective activator of a highly limited number of PPARγ pathways. Among these INT131-sensitive pathways are metabolic pathways including those pathways regulated by the hormone adiponectin.

As a result of this selective activation, administration of INT131 to patients results in fewer side effects than administration of other PPARγ agonists. For example, INT131 was equally efficacious in reducing HbA1c levels as 45 mg of pioglitazone but subjects taking INT131 experienced less edema, weight gain, and hemodilution than those taking pioglitazone. See, DePaoli, et al. Diabetes Care. 2014 Jul;37(7):1918-23. Thus, INT131 can administered to treat PSP while limiting side effects. Limiting side effects is advantageous as it helps preserve the quality of life for subject taking the medication and results in improved subject compliance with taking medication.

In particular, the invention provides a method of treating progressive supranuclear palsy or symptoms thereof in a subject in need thereof comprising administering to the subject a pharmaceutical composition comprising a therapeutically effective amount of a compound of formula (I),

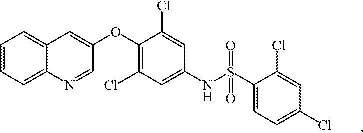

or a pharmaceutically acceptable salt, prodrug, or isomer thereof.

In one embodiment, the compound of formula (I) (i.e., INT131) is provided in the form of a besylate salt.

In one embodiment, the therapeutically effective amount is from about 0.1 to about 15 mg. In another embodiment, the therapeutically effective amount is from about 1 to about 10 mg. In still another embodiment, the therapeutically effective amount is from about 2 to about 6 mg. In yet another embodiment, the therapeutically effective amount is about 3 mg. In another embodiment, the therapeutically effective amount is about 15 mg, about 14 mg, about 13 mg, about 12 mg, about 11 mg, about 10 mg, about 9 mg, about 8 mg, about 7 mg, about 6 mg, about 5 mg, about 4 mg, about 3 mg, about 2 mg, or about 1 mg.

The pharmaceutical compositions used in the methods of the invention may be administered to the subject twice a day, daily, every other day, three times a week, twice a week, weekly, every other week, twice a month, or monthly.

Preferably, the methods of the invention result in increase of the adiponectin level in the subject by at least about 30%, at least about 68%, at least about 175%, or at least about 200%.

Provided herein are methods of treating a subject that include: selecting a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and administering a pharmaceutical composition including a therapeutically effective amount of a compound of formula (I),

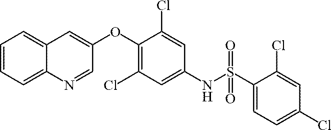 [SMILES: O=S(=O)(Nc1cc(Cl)c(Oc2cnc3ccccc3c2)c(Cl)c1)c1ccc(Cl)cc1Cl]

or a pharmaceutically acceptable salt, prodrug, or isomer thereof, to the selected subject.

Also provided herein are methods of selecting a treatment for a subject that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject. Some embodiments of any of the methods described herein further include administering the selected treatment to the identified subject.

Also provided herein are methods of selecting a subject for treatment that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for treatment with a pharmaceutical composition comprising a therapeutically effective amount of a compound of formula (I), or a pharmaceutically acceptable salt, prodrug, or isomer thereof.

Also provided herein are methods of selecting a subject for participation in a clinical trial that include: identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that includes administration of a pharmaceutical composition including a therapeutically effective amount of a compound of formula (I), or a pharmaceutically acceptable salt, prodrug, or isomer thereof.

Also provided herein are methods of predicting the efficacy of a treatment in a subject that include: determining a level of neurofilament light chain protein level in a sample including cerebrospinal fluid, blood, serum, or plasma obtained from the subject; and determining that a treatment with a pharmaceutical composition including a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein (e.g., any of the exemplary reference levels of neurofilament light chain protein described herein), as compared to a subject not having an elevated level of neurofilament light chain protein in a sample including blood, serum, or plasma as compared to the reference level of neurofilament light chain protein.

In some embodiments of any of the methods described herein, the subject has not been diagnosed with a neurological disorder or neural tissue damage. In some embodiments of any of the methods described herein, the subject does not present with a symptom of a neurological disorder or neural tissue damage (e.g., any of the symptoms of a neurological disorder or neural tissue damage described herein or known in the art). In some embodiments of any of the methods described herein, the subject has been diagnosed as having a neurological disorder (e.g., any of the neurological disorders described herein or known in the art) or neural tissue damage. In some embodiments of any of the methods described herein, the subject has been diagnosed as having progressive supranuclear palsy. Some embodiments of any of the methods described herein further include performing an assay to determine the level of neurofilament light chain protein in the sample obtained from the subject (e.g., any of the exemplary assays for determining a level of neurofilament light chain protein described herein or known in the art). In some embodiments of any of the methods described herein, the assay is a single-molecule array assay.

In some embodiments of any of the methods described herein, the subject has been previously administered a different pharmaceutical composition and the different pharmaceutical composition was determined not to be therapeutically effective.

Also provided herein are methods of determining the efficacy of a treatment in a subject that include: determining a first level of neurofilament light chain protein level in a sample including cerebrospinal fluid, blood, serum, or plasma obtained from the subject at a first time point; determining a second level of neurofilament light chain protein level in a sample including cerebrospinal fluid, blood, serum, or plasma obtained from the subject at a second time point, where the subject received at least one dose of a pharmaceutical composition including a therapeutically effective amount of a compound of formula (I), or a pharmaceutically acceptable salt, prodrug, or isomer thereof, between the first and the second time points; and identifying the pharmaceutical composition as being effective in a subject having a reduced second level of neurofilament light chain protein as compared to the first level of neurofilament light chain protein.

In some embodiments of any of the methods described herein, the subject is a participant in a clinical trial. Some embodiments of any of the methods described herein, the method further includes administering one or more additional doses of the pharmaceutical composition identified as being effective in the subject.

Provided herein are methods of treating a subject that includes: selecting a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subj ect.

Also provided herein are methods of selecting a treatment for a subject that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject. Some embodiments of any of the methods described herein further include administering the selected treatment to the identified subject.

Also provided herein are methods of selecting a subject for treatment that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject for participation in a clinical trial that include: identifying a subject having an elevated second level of neurofilament light chain protein in a sample providing cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for participation in a clinical trial that includes administration of a pharmaceutical composition including a therapeutically effective amount of a compound of formula (I), or a pharmaceutically acceptable salt, prodrug, or isomer thereof.

In some embodiments of any of the methods described herein, wherein the subject has not been diagnosed with a neurological disorder (e.g., any of the neurological disorders described herein or known in the art) or neural tissue damage. In some embodiments of any of the methods described herein, the subject does not present with a symptom of a neurological disorder or neural tissue damage (e.g., any of the symptoms of a neurological disorder or neural tissue damage described herein or known in the art). In some embodiments of any of the methods described herein, the subject has been diagnosed as having a neurological disorder (e.g., any of the neurological disorders described herein or known in the art) or neural tissue damage. In some embodiments of any of the methods described herein, the subject has been diagnosed as having progressive supranuclear palsy.

Some embodiments of any of the methods described herein that includes performing an assay to determine the first level and second levels of neurofilament light chain protein in the sample obtained from the subject at the first time point and the second time point, respectively (e.g., any of the assays for determining a level of neurofilament light chain protein described herein or known in the art). In some embodiments of any of the methods described herein, the assay is a single-molecule array assay.

In some embodiments of any of the methods described herein, the subject has been previously administered a different pharmaceutical composition and the different pharmaceutical composition was determined not to be therapeutically effective. In some embodiments of any of the methods described herein, the subject has not been diagnosed with a neurological disorder (e.g., any of the neurological disorders described herein or known in the art) or neural tissue damage. In some embodiments of any of the methods described herein, the subject does not present with a symptom of a neurological disorder or neural tissue damage (e.g., any of the symptoms of a neurological disorder or neural tissue damage described herein or known in the art).

### BRIEF DESCRIPTION OF THE DRAWINGS

FIG. 1. Serum adiponectin levels at Day 1, 4, 8, and 14 following administration of a placebo or 0.1, 1.0 or 4.0 mg INT131.

FIG. 2. Neuroprotective effects of CHS-131: subjects with a neurodegenerative disease (RRMS) administered 3 mg daily CHS-131 for six months had 34.2% less cortical volume loss and 50% less whole brain volume loss, when compared to placebo.

### DETAILED DESCRIPTION OF THE INVENTION

In particular, the compound (I),

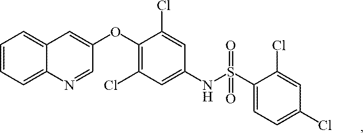

has been found to be unexpectedly effective for the treatment of progressive supranuclear palsy. This compound is also known as INT131 or CHS 131.

### Definitions

The terms “treat,” “treating” and “treatment” refer to a method of alleviating or abrogating a disease and/or its attendant symptoms. In another embodiment, treating refers to impeding or halting progression of a disease. In yet another embodiment, treating refers to extending the life of a subject with a disease.

In some embodiments, treatment can result in a reduction (e.g., an about 1% to about 99% reduction, an about 1% to about 95% reduction, an about 1% to about 90% reduction, an about 1% to about 85% reduction, an about 1% to about 80% reduction, an about 1% to about 75% reduction, an about 1% to about 70% reduction, an about 1% to about 65% reduction, an about 1% to about 60% reduction, an about 1% to about 55% reduction, an about 1% to about 50% reduction, an about 1% to about 45% reduction, an about 1% to about 40% reduction, an about 1% to about 35% reduction, an about 1% to about 30% reduction, an about 1% to about 25% reduction, an about 1% to about 20% reduction, an about 1% to about 15% reduction, an about 1% to about 10% reduction, an about 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, or an about 15% to about 20% reduction) in the number, severity, and/or duration of one or more (e.g., two, three, four, five, or six) symptoms and/or metrics (e.g., scores) of a neurological disorder (e.g., progressive supranuclear palsy) or neural tissue damage (e.g., any of the symptoms and/or metrics of any of the neurological disorders described herein or known in the art) (e.g., any of the symptoms and/or metrics of progressive supranuclear palsy described herein or known in the art).

The term “progressive supranuclear palsy” or “PSP” refers to a neurologic disorder of unknown origin that gradually destroys cells in many areas of the brain and the accumulation of abnormal aggregates of the microtubule-associated protein tau, resulting in insoluble paired helical filaments [Friedhoff, 2000 #12095], including the gradual deterioration of neurons and glial cells in the midbrain and frontal cortex that display insoluble helical filaments of tau proteins.

PSP starts with a pre-symptomatic phase during which there is an increase in neuropathological abnormalities. Next, patients develop isolated symptoms that are suggestive of PSP (soPSP). In any of the methods described herein, PSP can be classic PSP-Richardson’s syndrome (PSP-RS), PSP-Parkinsonism (PSP-P), PSP-corticobasal syndrome (PSP-CBS), PSP-progressive non-fluent aphasia (PSP-PNFA), or PSP-pure akinesia with gait freezing (PSP-PAGF) (Ling et al., J. Mov. Discord. 9(1):3-13, 2016). In some embodiments of any of the methods described herein, a subject can be previously diagnosed or identified as having PSP (e.g., PSP-RS, PSP-P, PSP-PNFA, or PSP-PAGF). In some embodiments of any of the methods described herein, a subject can previously be identified as having an increased risk of developing PSP (e.g., a subject having a genetically-related family member (e.g., a parent, grandparent, aunt, uncle, or sibling) that has been identified or diagnosed as having PSP). In some embodiments of any of the methods described herein, a subject can previously be identified or diagnosed as having pre-symptomatic PSP or suggestive-of-PSP.

After onset, symptoms of PSP become rapidly and progressively worse. Subjects diagnosed with PSP may become severely disabled within five years and die within six years.

Symptoms of PSP usually first appear at the age of 60 and worsen until death. People with PSP commonly die from pneumonia, choking or other complications caused by the loss of functional brain cells, resulting in loss of autonomic and motor function (e.g. the ability to swallow).

Signs and symptoms of PSP include movement, cognitive and psychiatric disorders. Voluntary movement can be impaired in PSP and include pseudobulbar palsy (i.e. inability to control facial movements), bradykinesia (i.e. slow or abnormal muscle movement), neck and trunk rigidity, impaired gait, impaired balance, posture instability and difficulty with speech and swallowing. Individuals who become unable to swallow food can be fitted with a feeding tube to provide nutriention. A most obvious, outward sign of the disease is an inability to coordinate and move the eyes normally, resulting in a vertical gaze palsy. Cognitive impairments include loss of executive functions (e.g. attention control, inhibitory control, working memory, cognitive flexibility, reasoning, problem solving and planning) and diminished fluency. Associated psychiatric symptoms include depression, feelings of irritability, sadness or apathy, insomnia, fatigue and loss of energy.

In some embodiments, a subject can be identified as having PSP using the MDS PSP Diagnostic Criteria (as described in, e.g., Hoglinger et al., Mov. Disord. 31:644-652, 2016).

In some embodiments, a subject can be identified as being at increased risk of developing PSP or identified as having PSP (e.g., any of the types of PSP described herein), e.g., at least in part, by detecting a genetic alteration in a gene encoding the microtubule-associated protein tau (MAPT) (e.g., any of the inversion polymorphisms in the MAPT gene, any of the haplotype-specific polymorphisms in the MAPT gene, the rare-coding MAPT variant (A152T), or mutations that enhance splicing of exon 10 in the MAPT gene described, e.g., in Hoglinger et al., Nature Genet. 43:699-705, 2011, and Hinz et al., Cold Spring Harb. Perspect Biol.). Non-limiting examples of genetic alterations in a gene encoding MAPT include mutations that result in the production of MAPT protein that include one or more point mutations of: S285R, L284R, P301L, and G303V. Additional specific genetic mutations in a gene encoding MAPT protein that can be used to identify a subject as having an increased risk of developing PSP or can be used to identify a subject as having PSP (e.g., any of the types of PSP described herein) are described in, e.g., Boxer et al., Lancet 16:552-563, 2017.

In some embodiments, a subject can be identified as having an increased risk of developing PSP or identified as having PSP (e.g., any of the types of PSP described herein), e.g., at least in part, by detecting tau protein deposits (e.g., 4-repeat tau protein deposits), detecting of atrophy of the midbrain and/or superior cerebellar peduncles (e.g., using any of the imaging techniques described herein or known in the art, e.g., magnetic resonance imaging (MRI) or positron emission tomography (PET) scans), and/or detecting of hypometabolism in the frontal cortex, caudate, and/or thalamus in the subject (e.g., using any of the imaging techniques described herein or known in the art, e.g., MRI, CT scan, or PET scan).

For example, in some embodiments a subject can be identified or diagnosed as having PSP (e.g., any of the types of PSP described herein), e.g., at least in part, by using MRI to detect brain atrophy (Min et al., Nat. Med. 21:1154-1162, 2015; Yanamandra et al., Ann. Clin. Transl. Neurol.2:278-288, 2015), changes in regional gray and white matter volume to detect atrophy (see, e.g., Josephs et al.., Brain 137:2783-2795, 2014; Santos-Santos et al., JAMA Neurol. 73:733-742, 2016), and midbrain atrophy by detecting midbrain area and volume in the subject (Josephs et al., Neurobiol. Aging 29:280-289, 2008; Whitwell et al., Eur. J. Neurol.20:1417-1422, 2013). In some embodiments, a subject can be identified or diagnosed as having PSP (e.g., any of the types of PSP described herein), e.g., at least in part, by administering to a subject a tau protein tracer (e.g., AV1451 or PBB3) and detecting tau protein in the subject’s brain using a PET scan (see, e.g., Marquie et al., Ann. Neurol. 78:787-800, 2015; Cho et al., Mov. Disord. 32:134-140, 2017; Whitwell et al., Mov. Disord. 32:124-133, 2017; and Smith et al., Mov. Disord. 32:108-114, 2017). In some embodiments, a subject can be diagnosed or identified as having PSP (e.g., any of the types of PSP described herein), e.g., at least in part, by detecting the difference in binaural masking level in the subject using a PET scan (see, e.g., Hughes et al., J. Neurophysiol. 112:3086-3094, 2014).

In some embodiments, a subject can be identified as being at increased risk of developing PSP or identified as having PSP (e.g., any of the types of PSP described herein), e.g., at least in part, by detecting the presence of, or an elevated level (e.g., as compared to a level in a healthy control subject) of, one or more biomarkers in a subject. In some embodiments, a subject can be identified or diagnosed as having PSP (e.g., any of the types of PSP described herein), e.g., at least in part, by detecting the presence of, or detection of an elevated level (e.g., as compared to a level in a healthy control subject) of, neurofilament light chain in the blood and/or cerebrospinal fluid in a subject (e.g., using any of the immunoassays described in Scherling et al., Ann. Neurol. 75:116-126, 2014; Bacioglu et al., Neuron 91:56-66, 2016; and Rojas et al., Ann. Clin. Transl. Neurol. 3:216-255, 2016). In some embodiments, a subject can be identified or diagnosed as having PSP (e.g., any of the types of PSP described herein), e.g., at least in part, by detecting the presence of, or detection of an elevated level (e.g., as compared to a level in a healthy control subject) of, YKL-40 in cerebrospinal fluid from the subject (see, e.g., Magdalinou et al., J. Neurol. Neurosurg. Psychiatry 2014 Oct; 85(10): 1065-1075; and Magdalinou et al., J. Neurol. Neurosurg. Psychiatry 86:1240-1247, 2015).

In some embodiments, a subject can be identified or diagnosed as having PSP (e.g., any of the types of PSP described herein), e.g., at least in part, by detecting decreased saccade velocity and gain in the subject using infrared oculography (see, e.g., Boxer et al., Arch. Neurol. 69:509-517, 2012; Boxer et al., Lancet Neurol. 132:676-685, 2014). In some embodiments, a subject can be identified or diagnosed as having PSP (e.g., any of the types of PSP described herein), e.g., at least in part, by detecting a spontaneous and evoked blink rate associated with PSP in the subject (see, e.g., Bologna et al., Brain 132:502-510, 2009). In some embodiments, a subject can be identified or diagnosed as having PSP (e.g., any of the types of PSP described herein), e.g., at least in part, by detecting decreased retinal thickness in a subject’s eye using optical coherence tomography (see, e.g., Schneider et al., J. Neural Transm. 121:41-47, 2014). In some embodiments, a subject can be identified or diagnosed as having PSP (e.g., any of the types of PSP described herein), e.g., at least in part, by detecting disrupted circadian rhythms and sleep in the subject (see, e.g., Walsh et al., SleepMed. 22;50-56, 2016).

In some embodiments, a subject can be identified or diagnosed as having PSP (e.g., any of the types of PSP described herein), e.g., at least in part, by detected a decreased ratio of 33 kDa tau to 55 kDa tau in the subject (e.g., as compared to the ratio of 33 kDa tau to 55 kDa tau in a healthy subject). In some embodiments, a subject can be identified or diagnosed as having PSP (e.g., any of the types of PSP described herein), e.g., at least in part, by detecting the presence of, or detecting an elevated level of, protein tyrosine phosphatase 1 (Ptpn1) (e.g., as described in Santiago et al., Mov. Discord. 29(4):550-555, 2014).

Some embodiments of any of the methods described herein can include monitoring the progression of PSP in the subject, e.g., by assessing the severity of PSP in the subject over time, e.g., using the Progressive Supranuclear Palsy Rating Scale (PSPRS) (e.g., as described in Golbe et al., Brain 130(6): 1552-1565, 2007). The PSPRS evaluates subjects according to their ability to perform daily activities, behavior, bulbar function, ocular motor function, limb motor function, and gait.

In some embodiments of any of the methods described herein, a subject can be further administered levodopa and/or physical therapy (e.g., as described in Lamb et al., Curr. Treat Options Neurol. 14:42, 2016, and Clerici et al., PLoS One 12:e0170927, 2017).

The term “therapeutically effective amount” refers to that amount of the compound being administered sufficient to treat a disease. In one embodiment, the therapeutically effective amount is sufficient to prevent development of or alleviate to some extent one or more of the symptoms of the condition or disorder being treated.

The term “subject” is defined herein to include animals such as mammals, including but not limited to, primates (e.g., humans), cows, sheep, goats, horses, dogs, cats, rabbits, rats, mice and the like. In preferred embodiments, the subject is a human.

In some embodiments of any of the methods described herein, a subject may be referred to as a patient. In some embodiments of any of the methods described herein, the subject is 40 years old or older (e.g., 41 years old or older, 42 years old or older, 43 years old or older, 44 years old or older, 45 years old or older, 50 years old or older, 55 years old or older, 60 years old or older, 65 years old or older, 70 years old or older, 75 years old or older, 80 years old or older, 90 years old or older, or 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, or 103 years old).

In some embodiments, the subject does not present with a symptom (e.g., any of the symptoms described herein or known in the art) of a neurological disorder or neural tissue damage (e.g., progressive supranuclear palsy, multiple sclerosis, relapsing-remitting MS (RRMS), clinically isolated syndrome (CIS), primary progressive MS (PPMS), secondary progressive MS (SPMS), or radiologically isolated syndrome (RIS)). In other embodiments, the subject has been diagnosed as having a neurological disorder (e.g., multiple sclerosis, relapsing-remitting MS (RRMS), clinically isolated syndrome (CIS), primary progressive MS (PPMS), secondary progressive MS (SPMS), or radiologically isolated syndrome (RIS)) or neural tissue damage. In other embodiments, the subject has been diagnosed as having neural tissue damage (e.g., severe traumatic brain injury, sports-related mild traumatic brain injury, or post-concussion syndrome). In yet other embodiments, the subject has not been diagnosed as having a neurological disorder or neural tissue damage.

In some embodiments, the subject has a degenerative and traumatic neurological disorder (e.g., dementia, amyotrophic lateral sclerosis, or spinal cord injury).

In yet other embodiments, the subject has been diagnosed or identified as having a neurological disorder that would benefit from treatment with a proliferator-activated receptor gamma (PPARy) agonist (e.g., CHS-131).

In some embodiments, the subject has previously been administered at least one dose of a CHS-131. In some embodiments, the subject is a participant in a clinical trial.

In other embodiments, the subject has been previously administered a different pharmaceutical composition and the different pharmaceutical composition was determined not to be therapeutically effective.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either net or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either net or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isbutyric, oxalic, maleic, malonic, benzoic, succinic, suberic, fumeric mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present inventions contain both basic and acidic functionalities that allow the compounds to be converted into either base or acid addition salts.

The neutral forms of the compounds may be registered by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In additional to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, be bioavailable by oral administration whereas the parent drug is not. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity. Additional examples include peptidyl derivatives of a compound of the invention.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

As used herein, the term “biological sample” or “sample” refers to a sample obtained or derived from a subject. By way of example, the sample can include cerebrospinal fluid (CSF), blood, serum, or plasma. In some embodiments, a sample can be, or include, a blood sample. In some embodiments, a sample can be, or include, a serum sample. In some embodiments, a sample can be, or include, a plasma sample.

As used herein, “obtain” or “obtaining” can be any means whereby one comes into possession of the sample by “direct” or “indirect” means. Directly obtaining a sample means performing a process (e.g., performing a physical method such as extraction or phlebotomy) to obtain a sample from the subject. Indirectly obtaining a sample refers to receiving the sample from another party or source (e.g., a third-party laboratory that directly acquired the sample). Thus, obtain is used to mean collection and/or removal of the sample from the subject. Some embodiments of any of them methods described herein can include obtaining a sample or samples from a subject.

The phrase “an elevated” or “an increased level” can be an elevation or an increase of at least 1% (e.g., at least 2%, at least 4%, at least 6%, at least 8%, at least 10%, at least 12%, at least 14%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 110%, at least 115%, at least 120%, at least 140%, at least 150%, at least 200%, at least 250%, at least 300%, at least 350%, at least 400%, or between 1% and 400%, between 1% and 300%, between 1% and 200%, between 1% and 100%, between 1% and 50%, between 1% and 25%, between 1% and 10%, between 10% and 400%, between 10% and 300%, between 10% and 200%, between 10% and 100%, between 10% and 50%, between 50% and 400%, between 50% and 300%, between 50% and 200%, between 50% and 100%, between 50% and 75%, between 75% and 100%, or 1%, 2%, 4%, 5%, 6%, 8%, 10%, 12%, 14%, 16%, 18%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140% ,145%, 150%, 155%, 160%, 165%, 170%, 175%, 180%, 185%, 190%, 195%, 200%, 250%, 300%, 350%, or 400%), e.g., as compared to a reference level (e.g., any of the exemplary reference levels described herein) or a first level of neurofilament light chain protein.

In some embodiments, an elevated level can be an elevation or an increase of about 1% to about 500%, about 1% to about 450%, about 1% to about 400%, about 1% to about 350%, about 1% to about 300%, about 1% to about 250%, about 1% to about 200%, about 1% to about 150%, about 1% to about 100%, about 1% to about 50%, about 1% to about 25%, about 1% to about 20%, about 1% to about 15%, about 1% to about 10%, about 1% to about 5%, about 2% to about 500%, about 2% to about 450%, about 2% to about 400%, about 2% to about 350%, about 2% to about 300%, about 2% to about 250%, about 2% to about 200%, about 2% to about 150%, about 2% to about 100%, about 2% to about 50%, about 2% to about 25%, about 2% to about 20%, about 2% to about 15%, about 2% to about 10%, about 5% to about 500%, about 5% to about 450%, about 5% to about 400%, about 5% to about 350%, about 5% to about 300%, about 5% to about 250%, about 5% to about 200%, about 5% to about 150%, about 5% to about 100%, about 5% to about 50%, about 5% to about 25%, about 5% to about 20%, about 5% to about 15%, about 5% to about 10%, about 10% to about 500%, about 10% to about 450%, about 10% to about 400%, about 10% to about 350%, about 10% to about 300%, about 10% to about 250%, about 10% to about 200%, about 10% to about 150%, about 10% to about 100%, about 10% to about 50%, about 10% to about 25%, about 10% to about 20%, about 10% to about 15%, about 15% to about 500%, about 15% to about 450%, about 15% to about 400%, about 15% to about 350%, about 15% to about 300%, about 15% to about 250%, about 15% to about 200%, about 15% to about 150%, about 15% to about 100%, about 15% to about 50%, about 15% to about 25%, about 15% to about 20%, about 20% to about 500%, about 20% to about 450%, about 20% to about 400%, about 20% to about 350%, about 20% to about 300%, about 20% to about 250%, about 20% to about 200%, about 20% to about 150%, about 20% to about 100%, about 20% to about 50%, about 20% to about 25%, about 25% to about 500%, about 25% to about 450%, about 25% to about 400%, about 25% to about 350%, about 25% to about 300%, about 25% to about 250%, about 25% to about 200%, about 25% to about 150%, about 25% to about 100%, about 25% to about 50%, about 50% to about 500%, about 50% to about 450%, about 50% to about 400%, about 50% to about 350%, about 50% to about 300%, about 50% to about 250%, about 50% to about 200%, about 50% to about 150%, about 50% to about 100%, about 100% to about 500%, about 100% to about 450%, about 100% to about 400%, about 100% to about 350%, about 100% to about 300%, about 100% to about 250%, about 100% to about 200%, about 100% to about 150%, about 150% to about 500%, about 150% to about 450%, about 150% to about 400%, about 150% to about 350%, about 150% to about 300%, about 150% to about 250%, about 150% to about 200%, about 200% to about 500%, about 200% to about 450%, about 200% to about 400%, about 200% to about 350%, about 200% to about 300%, about 200% to about 250%, about 250% to about 500%, about 250% to about 450%, about 250% to about 400%, about 250% to about 350%, about 250% to about 300%, about 300% to about 500%, about 300% to about 450%, about 300% to about 400%, about 300% to about 350%, about 350% to about 500%, about 350% to about 450%, about 350% to about 400%, about 400% to about 500%, about 400% to about 450%, or about 450% to about 500%, e.g., as compared to a reference level (e.g., any of the exemplary reference levels described herein) or a first level of neurofilament light chain protein.

A “reduced level” can be a 1% to about 99% reduction, a 1% to about 95% reduction, a 1% to about 90% reduction, a 1% to about 85% reduction, a 1% to about 80% reduction, a 1% to about 75% reduction, a 1% to about 70% reduction, a 1% to about 65% reduction, a 1% to about 60% reduction, a 1% to about 55% reduction, a 1% to about 50% reduction, a 1% to about 45% reduction, a 1% to about 40% reduction, a 1% to about 35% reduction, a 1% to about 30% reduction, a 1% to about 25% reduction, a 1% to about 20% reduction, a 1% to about 15% reduction, a 1% to about 10% reduction, a 1% to about 5% reduction, an about 5% to about 99% reduction, an about 5% to about 95% reduction, an about 5% to about 90% reduction, an about 5% to about 85% reduction, an about 5% to about 80% reduction, an about 5% to about 75% reduction, an about 5% to about 70% reduction, an about 5% to about 65% reduction, an about 5% to about 60% reduction, an about 5% to about 55% reduction, an about 5% to about 50% reduction, an about 5% to about 45% reduction, an about 5% to about 40% reduction, an about 5% to about 35% reduction, an about 5% to about 30% reduction, an about 5% to about 25% reduction, an about 5% to about 20% reduction, an about 5% to about 15% reduction, an about 5% to about 10% reduction, an about 10% to about 99% reduction, an about 10% to about 95% reduction, an about 10% to about 90% reduction, an about 10% to about 85% reduction, an about 10% to about 80% reduction, an about 10% to about 75% reduction, an about 10% to about 70% reduction, an about 10% to about 65% reduction, an about 10% to about 60% reduction, an about 10% to about 55% reduction, an about 10% to about 50% reduction, an about 10% to about 45% reduction, an about 10% to about 40% reduction, an about 10% to about 35% reduction, an about 10% to about 30% reduction, an about 10% to about 25% reduction, an about 10% to about 20% reduction, an about 10% to about 15% reduction, an about 15% to about 99% reduction, an about 15% to about 95% reduction, an about 15% to about 90% reduction, an about 15% to about 85% reduction, an about 15% to about 80% reduction, an about 15% to about 75% reduction, an about 15% to about 70% reduction, an about 15% to about 65% reduction, an about 15% to about 60% reduction, an about 15% to about 55% reduction, an about 15% to about 50% reduction, an about 15% to about 45% reduction, an about 15% to about 40% reduction, an about 15% to about 35% reduction, an about 15% to about 30% reduction, an about 15% to about 25% reduction, an about 15% to about 20% reduction, an about 20% to about 99% reduction, an about 20% to about 95% reduction, an about 20% to about 90% reduction, an about 20% to about 85% reduction, an about 20% to about 80% reduction, an about 20% to about 75% reduction, an about 20% to about 70% reduction, an about 20% to about 65% reduction, an about 20% to about 60% reduction, an about 20% to about 55% reduction, an about 20% to about 50% reduction, an about 20% to about 45% reduction, an about 20% to about 40% reduction, an about 20% to about 35% reduction, an about 20% to about 30% reduction, an about 20% to about 25% reduction, an about 25% to about 99% reduction, an about 25% to about 95% reduction, an about 25% to about 90% reduction, an about 25% to about 85% reduction, an about 25% to about 80% reduction, an about 25% to about 75% reduction, an about 25% to about 70% reduction, an about 25% to about 65% reduction, an about 25% to about 60% reduction, an about 25% to about 55% reduction, an about 25% to about 50% reduction, an about 25% to about 45% reduction, an about 25% to about 40% reduction, an about 25% to about 35% reduction, an about 25% to about 30% reduction, an about 30% to about 99% reduction, an about 30% to about 95% reduction, an about 30% to about 90% reduction, an about 30% to about 85% reduction, an about 30% to about 80% reduction, an about 30% to about 75% reduction, an about 30% to about 70% reduction, an about 30% to about 65% reduction, an about 30% to about 60% reduction, an about 30% to about 55% reduction, an about 30% to about 50% reduction, an about 30% to about 45% reduction, an about 30% to about 40% reduction, an about 30% to about 35% reduction, an about 35% to about 99% reduction, an about 35% to about 95% reduction, an about 35% to about 90% reduction, an about 35% to about 85% reduction, an about 35% to about 80% reduction, an about 35% to about 75% reduction, an about 35% to about 70% reduction, an about 35% to about 65% reduction, an about 35% to about 60% reduction, an about 35% to about 55% reduction, an about 35% to about 50% reduction, an about 35% to about 45% reduction, an about 35% to about 40% reduction, an about 40% to about 99% reduction, an about 40% to about 95% reduction, an about 40% to about 90% reduction, an about 40% to about 85% reduction, an about 40% to about 80% reduction, an about 40% to about 75% reduction, an about 40% to about 70% reduction, an about 40% to about 65% reduction, an about 40% to about 60% reduction, an about 40% to about 55% reduction, an about 40% to about 50% reduction, an about 40% to about 45% reduction, an about 45% to about 99% reduction, an about 45% to about 95% reduction, an about 45% to about 90% reduction, an about 45% to about 85% reduction, an about 45% to about 80% reduction, an about 45% to about 75% reduction, an about 45% to about 70% reduction, an about 45% to about 65% reduction, an about 45% to about 60% reduction, an about 45% to about 55% reduction, an about 45% to about 50% reduction, an about 50% to about 99% reduction, an about 50% to about 95% reduction, an about 50% to about 90% reduction, an about 50% to about 85% reduction, an about 50% to about 80% reduction, an about 50% to about 75% reduction, an about 50% to about 70% reduction, an about 50% to about 65% reduction, an about 50% to about 60% reduction, an about 50% to about 55% reduction, an about 55% to about 99% reduction, an about 55% to about 95% reduction, an about 55% to about 90% reduction, an about 55% to about 85% reduction, an about 55% to about 80% reduction, an about 55% to about 75% reduction, an about 55% to about 70% reduction, an about 55% to about 65% reduction, an about 55% to about 60% reduction, an about 60% to about 99% reduction, an about 60% to about 95% reduction, an about 60% to about 90% reduction, an about 60% to about 85% reduction, an about 60% to about 80% reduction, an about 60% to about 75% reduction, an about 60% to about 70% reduction, an about 60% to about 65% reduction, an about 65% to about 99% reduction, an about 65% to about 95% reduction, an about 65% to about 90% reduction, an about 65% to about 85% reduction, an about 65% to about 80% reduction, an about 65% to about 75% reduction, an about 65% to about 70% reduction, an about 70% to about 99% reduction, an about 70% to about 95% reduction, an about 70% to about 90% reduction, an about 70% to about 85% reduction, an about 70% to about 80% reduction, an about 70% to about 75% reduction, an about 75% to about 99% reduction, an about 75% to about 95% reduction, an about 75% to about 90% reduction, an about 75% to about 85% reduction, an about 75% to about 80% reduction, an about 80% to about 99% reduction, an about 80% to about 95% reduction, an about 80% to about 90% reduction, an about 80% to about 85% reduction, an about 85% to about 99% reduction, an about 85% to about 95% reduction, an about 85% to about 90% reduction, an about 90% to about 99% reduction, an about 90% to about 95% reduction, or an about 95% to about 99% reduction, e.g., in a second level of neurofilament light chain protein as compared to a first level of neurofilament light chain protein.

As used herein a “first time point” can, e.g., refer to an initial time point wherein the subject has not yet received a dose of a pharmaceutical composition (e.g., any of the pharmaceutical compositions described herein). In some embodiments, a first time point can be, e.g., a time point when a subject has been diagnosed with a neurological disorder or neural tissue damage prior to receiving any treatment (e.g., any of the exemplary treatments described herein). In other examples, a first time point can be a time point when a subject has developed at least one (e.g., 1, 2, 3, 4, 5, 6, 7, 8, 9 or 10) symptom(s) associated with a neurological disorder or neural tissue damage (e.g., any of the exemplary symptoms of a neurological disorder or neural tissue damage described herein or known in the art) and has not received any treatment. In some embodiments, a first time point can represent a time point after which a subject has previously received a different pharmaceutical treatment and the different pharmaceutical treatment was deemed not be successful.

As used herein a “second time point” can, e.g., refer to a second time point after the first time point. In some embodiments, a subject can receive or has received at least one (e.g., 1, 2, 3, 4, 5, 6, 7, 8, 9 or 10) dose of a pharmaceutical composition (e.g., any of the pharmaceutical compositions) between the first and the second time points. In some embodiments, the time difference between a first and second time point can be, e.g., 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, 42 days, 43 days, 44 days, 45 days, 46 days, 47 days, 48 days, 49 days, 50 days, 51 days, 52 days, 53 days, 54 days, 55 days, 56 days, 57 days, 58 days, 59 days, 60 days, 61 days, 62 days, 63 days, 64 days, 65 days, 66 days, 67 days, 68 days, 69 days, 70 days, 71 days, 72 days, 73 days, 74 days, 75 days, 76 days, 77 days, 78 days, 79 days, 80 days, 81 days, 82 days, 83 days, 84 days, 85 days, 86 days, 87 days, 88 days, 89 days, 90 days, 91 days, 92 days, 93 days, 94 days, 95 days, 96 days, 97 days, 98 days, 99 days, 100 days, 101 days, 102 days, 103 days, 104 days, 105 days, 106 days, 107 days, 108 days, 109 days, 110 days, 111 days, 112 days, 113 days, 114 days, 115 days, 116 days, 117 days, 118 days, 119 days, 120 days, 121 days, 122 days, 123 days, 124 days, 125 days, 126 days, 127 days, 128 days, 129 days, 130 days, 131 days, 132 days, 133 days, 134 days, 135 days, 136 days, 137 days, 138 days, 139 days, 140 days, 141 days, 142 days, 143 days, 144 days, 145 days, 146 days, 147 days, 148 days, 149 days, 150 days, 151 days, 152 days, 153 days, 154 days, 155 days, 156 days, 157 days, 158 days, 159 days, 160 days, 161 days, 162 days, 163 days, 164 days, 165 days, 166 days, 167 days, 168 days, 169 days, 170 days, 171 days, 172 days, 173 days, 174 days, 175 days, 176 days, 177 days, 178 days, 179 days, 180 days, 7 months, 8 months 9 months, 10 months, 11 months, or 1 year.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### EMBODIMENTS OF THE INVENTION

A new use of a known compound that modulates PPARγ has now been discovered. Specifically, it has been discovered that PPARγ agonists, and in particular, INT131, are effective to treat progressive supranuclear palsy.

Thus, in one embodiment, the present invention is directed to a method of treating progressive supranuclear palsy or its symptoms in a subject in need thereof comprising administering to the subject a pharmaceutical composition comprising a therapeutically effective amount of INT131 or a pharmaceutically acceptable salt, prodrug, or isomer thereof.

Without wishing to be limited to a particular theory, it is believed that INT131 increases PPARy activation in brain cells (including activation of elements in the PPARγ pathway), increases adiponectin levels, improves energy metabolism in brain cells, and reduces or prevents aggregation of abnormal Tau proteins by reducing oxidative stress and neuroinflammation, and therefore, treats progressive supranuclear palsy.

Adiponectin affects glucose and lipid metabolism, insulin sensitivity, and inflammation. Yamauchi T, et al., Adiponectin stimulates glucose utilization and fatty-acid oxidation by activating AMP-activated protein kinase. Nat Med . 2002;8:1288-1295; Berg AH, et al., The adipocyte-secreted protein Acrp30 enhances hepatic insulin action. Nat Med . 2001;7:947-953.; and Yamauchi T, et al., Targeted disruption of AdipoR1 and AdipoR2 causes abrogation of adiponectin binding and metabolic actions. Nat Med. 2007;13:332-339. Additionally, adiponectin targets the central nervous system and it also modulates appetite and energy homeostasis. Moreover, adiponectin stimulates food intake. Kubota N, et al., Adiponectin stimulates AMP-activated protein kinase in the hypothalamus and increases food intake. Cell Metab. 2007;6:55-68. Since subjects with progressive supranuclear palsy suffer weight loss, administration of INT131 to increase adiponectin and stimulate food intake treats subjects with progressive supranuclear palsy.

PSP has been associated with increased oxidative stress and neuroinflammation. Specifically, malondialdehyde, a biomarker for oxidative stress, has been demonstrated to be elevated in neurons in areas of the brain affected by PSP, but not in unaffected areas. Albers DS, et al., Frontal lobe dysfunction in progressive supranuclear palsy: evidence for oxidative stress and mitochondrial impairment, JNeurochem. 2000 Feb;74(2):878-81. Further, the cortex of PSP patients show a 39% decrease in ketoglutarate dehydrodenase/glutamate dehydrogenase ratio, which is indicative of oxidative stress to those areas. Albers, et al. 2000. Finally, patients affected with PSP demonstrate abnormal lipid peroxidation and protein peroxidation, hallmarks of oxidative stress. Odetti P, et al., Lipoperoxidation is selectively involved in progressive supranuclear palsy, J NeuropatholExp Neurol. 2000 May;59(5):393-7.

Activated microglia and astrocytes, which are associated with neuroinflammation, have been found in affected areas of the brain in PSP patients. Ferrer I, et al., Phosphorylated map kinase (ERK1, ERK2) expression is associated with early tau deposition in neurons and glial cells, but not with increased nuclear DNA vulnerability and cell death, in Alzheimer disease, Pick’s disease, progressive supranuclear palsy and corticobasal degeneration, Brain Pathol. 2001 Apr;11(2):144-58. Recent studies have shown a region-specific upregulation of IL-1β in the substantia nigra of PSP patients, as compared to either Parkinson’s disease or Alzheimer’s Disease brains. Moreover, Further other markers of neuroinflammation , including TNFα, and IL-6 have been found to be elevated in affected areas of the brain in PSP patients, whereas the anti-inflammatory cytokines TGFβ and IL-10 are not elevated. Fernandez-Botran R, et al., Cytokine expression and microglial activation in progressive supranuclear palsy, Parkinsonism Relat Disord. 2011 Nov;17(9):683-8.

PPARy agonists, including INT131, have been shown to inhibit both oxidative stress and neuroinflammation in animal models and humans. Specifically, this inhibition has been demonstrated for: pioglitazone and rosiglitazone for ischemic stroke with reperfusion, Collino M, et al., Modulation of the oxidative stress and inflammatory response by PPAR-gamma agonists in the hippocampus of rats exposed to cerebral ischemia/reperfusion, Eur J Pharmacol. 2006 Jan 13;530(1-2):70-80.; pioglitazone and rosiglitazone for traumatic brain injury, Kapadia R, et al., Mechanisms of anti-inflammatory and neuroprotective actions of PPAR-gamma agonists, Front Biosci. 2008 Jan 1;13:1813-26; INT131 for type-2 diabetes mellitus, Lee, DH, et al., Selective PPARγ modulator INT131 normalizes insulin signaling defects and improves bone mass in diet-induced obese mice. Am J PhysiolEndocrinol Metab. 2012;302:552-560; and INT131 for multiple sclerosis, U.S. Pat. Application Publication 2014/0213612.

Notably, there are disadvantages to treating humans with rosiglitazone and pioglitazone. Avandia (rosiglitazone) is only approved for treating patients with type 2 diabetes and increases the risk of: heart failure, cardiovascular events in individuals with heart failure, edema, weight gain, macular edema, bone fractures, decreases in hemoglobin and hemocrit, and other adverse events. Moreover, Avandia does not cross the blood-brain-barrier. Avanida package insert, September 2016. Since only 9-14% of rosiglitazone crosses the blood brain barrier, it may have limited efficacy or require more frequent and higher doses to adequately treat neurological disorders such as progressive supranuclear palsy. Like Avandia, Actos (pioglitazone) is only approved for treating patients with type 2 diabetes. Actos also caries several serious warnings and precautions including: increased risk of fluid retention leading to congestive heart failure, hypoglycemia, sometimes fatal hepatic failure, bladder cancer, edema, bone fractures, macular edema, and other adverse events. Actos package insert, December 2016. Notably, unlike the two commercially available PPAR-γ ligands, rosiglitazone and pioglitazone, INT131 readily crosses the blood brain barrier.

Despite data for full PPARy agonists decreasing oxidative stress and neuroinflammation, the benefits of INT131 is surprising since it was unknown if the selective PPARγ pathway activation of INT131 would treat PSP.

Accordingly, it is surprising and unexpected that INT131 treats progressive supranuclear palsy.

In one embodiment, INT131 reduces the signs and symptoms of progressive supranuclear palsy. INT131 reduces one or more sign or symptom of progressive supranuclear palsy. In a further embodiment, the reduction of the signs and symptoms of progressive supranuclear palsy is reduction in the number of the signs and symptoms of progressive supranuclear palsy. In yet a further embodiment, the reduction of the signs and symptoms of progressive supranuclear palsy is reduction in the severity or degree of at least one sign or symptom of progressive supranuclear palsy. In still a further embodiment, the reduction of the signs and symptoms of progressive supranuclear palsy is reduction in the duration of at least one sign or symptom of progressive supranuclear palsy.

In another embodiment, the onset of a sign or symptom of progressive supranuclear palsy is prevented or delayed in a subject with progressive supranuclear palsy upon administration of INT131 to the subject with progressive supranuclear palsy

In one embodiment, INT131 reduces neural inflammation in a subject with progressive supranuclear palsy.

In one embodiment, INT131 is neuroprotective in a subject with progressive supranuclear palsy. In another embodiment, INT131 treats neuronal degeneration. In another embodiment, INT131 reduces atrophy or degeneration of the brain in subjects with progressive supranuclear palsy. In a further embodiment, INT131 reduces atrophy or degradation of the substantial nigra, globus palladus, subthalamic nucleus and or cerebellum.

In one embodiment, INT131 reduces or prevents weight loss in a subject with progressive supranuclear palsy. In another embodiment, INT131 increases appetite in a subject with progressive supranuclear palsy.

In another embodiment, INT131 treats metabolic dysfunction in a subject with progressive supranuclear palsy. In yet a further embodiment, INT131 increases adiponectin levels in a subject with progressive supranuclear palsy. In another embodiment, INT131 improves or increases glucose metabolism in a subject with progressive supranuclear palsy. In another embodiment, INT131 reduces hyperglycemia in a subject with progressive supranuclear palsy.

In another embodiment, INT131 improves mitochondrial function. In a further embodiment, INT131 improves mitochondrial calcium handling, and mitochondrial trafficking. In yet a further embodiment, INT131 increases the expression of peroxisome proliferator-activated receptor gamma coactivator 1-alpha (PGC-1α) and mitochondrial biogenesis. This results in improved behavior, improved survival (i.e. lifespan) and reduced brain degeneration in a subject with progressive supranuclear palsy.

In another embodiment, INT131 reduces the aggregation of abnormal Tau protein, or fragments of Tau protein, in a subject with progressive supranuclear palsy. In a further embodiment, INT131 ameliorates the reduction of neuroprotective proteins in the brain. In yet a further embodiment, INT131 reduces the reduction of brain-derived neurotrophic factor and Bcl 2.

In another embodiment, a period of time during which the therapeutic effects of INT131 are observed in a subject with progressive supranuclear palsy comprises 1 week, 2 weeks, 3 weeks, 4 weeks, 1 month, 2 months, 3 months, 4 months, 5 months, 6 months, 7 months, 8 months, 9 months, 10 months, 11 months, 1 year, 1.25 years, 1.5 years, 1.75 years, 2 years, 2.25 years, 2.5 years, 2.75 years, 3 years, 3.5 years, 4 years, 4.5 years, and 5 years.

In one embodiment, INT131 is in the form of a besylate salt.

In another embodiment, the therapeutically effective amount is from about 0.1 to about 10 milligrams. In another embodiment, the therapeutically effective amount is from about 1 to about 4 milligrams. In another embodiment, the therapeutically effective amount is from about 2 to about 3 milligrams.

In another embodiment, a composition comprising a therapeutically effective amount of INT131 is administered to a subject in need thereof at an interval that includes, but is not limited to, twice a day, daily, every other day, three times a week, twice a week, weekly, every other week, twice a month, monthly, and every other month.

In another embodiment, a composition comprising a therapeutically effective amount of INT131 is administered orally to a subject. In yet another embodiment, the composition is substantially the same as those disclosed in US Patent Application Publication 2013-0243865, the disclosure of which is expressly incorporated herein by reference.

In one embodiment, INT-131 is as effective, or more effective, treating progressive supranuclear palsy than other therapies. These therapies include therapies approved for treating progressive supranuclear palsy and those in development for treating progressive supranuclear palsy. These therapies include, but are not limited to, medications to treat movement disorders, medications to treat psychiatric disorders, psychotherapy, speech therapy, physical therapy, and occupational therapy.

Medications to treat movement disorders include, but are not limited to, tetrabenazine, antipsychotic drugs, such as haloperidol, chlorpromazine, risperidone, and quetiapine, and other medications such as amantadine, levetiracetam, and, clonazepam.

Medications to treat psychiatric disorders include, but are not limited to, antidepressants such as citalopram, fluoxetine, and sertraline, antipsychotic drugs such as quetiapine, risperidone, and olanzapine, and mood-stabilizing drugs, including anticonvulsants, such as valproate, carbamazepine, and lamotrigine.

Psychotherapy includes, but is not limited to, talk therapy to help a subject manage behavioral problems, depression, and suicidal thoughts.

Speech therapy includes, but is not limited to, improving a subjects ability to speak clearly, and improve function and control of muscles used for eating and swallowing.

Physical therapy includes, but is not limited to, enhancing strength, flexibility, balance and coordination, reducing the risk of falls, and improve posture to lessen the severity of movement problems.

Occupational therapy includes, but is not limited to, use of assistive devices that improve functional abilities such as handrails, and eating and drinking utensils for subjects with diminished motor skills.

In another embodiment, INT-131 is administered to a subject in need thereof in combination with one or more therapies listed herein.

Also provided herein are methods of treating a subject that include: selecting a subject identified or diagnosed as having PSP (e.g., any of the types of PSP described herein) (e.g., using any of the techniques for identifying or diagnosing PSP described herein) and administering a therapeutically effective dose of a compound of formula (I), or a pharmaceutically acceptable salt, prodrug, or isomer thereof, to the selected subject. In some embodiments of these methods, the subject identified or diagnosed as having PSP did not benefit from a previously administered (different) therapeutic treatment for PSP.

Also provided herein are methods of treating a subject that include: selecting a subject identified as having an increased risk of developing PSP (e.g., any of the types of PSP described herein) (e.g., using any of the techniques for identifying a subject having an increased risk of developing PSP described herein) and administering a therapeutically effective dose of a compound of formula (I), or a pharmaceutically acceptable salt, prodrug, or isomer thereof, to the selected subject.

Also provided herein are methods of treating a subject that include: diagnosing or identifying a subject as having PSP (e.g., any of the types of PSP described herein) or having an increased risk of developing PSP (e.g., any of the types of PSP described herein) (e.g., by performing any of the techniques for diagnosing or identifying a subject as having PSP described herein or known in the art, or by performing any of the techniques for identifying a subject as having an increased risk of developing PSP described herein or known in the art), and administering a therapeutically effective dose of a compound of formula (I), or a pharmaceutically acceptable salt, prodrug, or isomer thereof, to the diagnosed or identified subj ect.

In some embodiments of any of the methods of treatment described herein, the method can result in a decreased risk of developing comorbidity in the subject (e.g., as compared to the risk of developing comorbidity in a similar subject having a similar neurological disorder (e.g., PSP), but administered a different treatment). Some embodiments of any of the methods described herein can further include administering to the subject an agent that alleviates a negative side effect of a compound of formula (I), or a pharmaceutically acceptable salt, prodrug, or isomer thereof, in the subject (e.g., weight loss or mood swings).

In some embodiments of any of the methods of treatment described herein, the method can result in increasing the life span of the subject (e.g., as compared to a similar subject having a similar neurological disorder (e.g., PSP), but receiving a different treatment). In some embodiments of any of the methods of treatment described herein, the method results in an improvement in the motor function of the subject (e.g., as compared to the motor function of the subject prior to treatment).

Some embodiments of any of the methods of treatment described herein further can include administering to the subject an agent for treating depression, obsessive-compulsive behavior, and/or apathy. Some embodiments of any of the methods described herein can further include administering to the subject an agent that alleviates eye irritation and/or eye closure symptoms. Some embodiments of any of the methods described herein can further include administering to the subject a treatment for reducing weight loss or a treatment for reducing the risk of developing aspiration pneumonia.

Also provided are methods of reducing the rate of progression of PSP in a subject (e.g., as compared to a similar subject having a similar stage of PSP but administered a different treatment) that include: identifying a subject having an early stage of PSP (e.g., pre-symptomatic PSP or suggestive-of-PSP) (e.g., by performing any of the techniques for determining the stage of PSP described herein); and administering a therapeutically effective dose of a compound of formula (I), or a pharmaceutically acceptable salt, prodrug, or isomer thereof, to the identified subj ect.

Also provided are methods of reducing the rate or progression of PSP in a subject (e.g., as compared to a similar subject having a similar stage of PSP but administered a different treatment) that include administering a therapeutically effective dose of a compound of formula (I), or a pharmaceutically acceptable salt, prodrug, or isomer thereof, to a subject identified or diagnosed as having an early state of PSP (e.g., pre-symptomatic PSP or suggestive-of-PSP) (e.g., using any of the techniques described herein).

Also provided herein are methods of selecting a treatment for a subject that include: selecting a subject identified or diagnosed as having PSP (e.g., any of the types of PSP described herein) (e.g., using any of the techniques for identifying or diagnosing PSP in a subject described herein), and selecting a therapeutically effective dose of a compound of formula (I), or a pharmaceutically acceptable salt, prodrug, or isomer thereof, for the selected subject.

Also provided herein are methods of selecting a subject for treatment with a compound of formula (I), or a pharmaceutically acceptable salt, prodrug, or isomer thereof, that include: selecting a subject identified as having an increased risk of developing PSP (e.g., any of the types of PSP described herein) (e.g., using any of the techniques for identifying a subject as having an increased risk of developing PSP described herein or known in the art); and selecting the selected subject for treatment with a therapeutically effective dose of a compound of formula (I), or a pharmaceutically acceptable salt, prodrug, or isomer thereof.

Also provided herein are methods of determining the efficacy of treatment in a subject having PSP that include: (a) determining a first level of one or more biomarkers (e.g., by performing an immunoassay) in a sample obtained from the subject at a first time point; (b) administering a therapeutically effective amount of a compound of formula (I), or a pharmaceutically acceptable salt, prodrug, or isomer thereof to the subject after the first time point; and (c) determining a second level of the one or more biomarkers (e.g., by performing an immunoassay) in a sample obtained from the subject at a second time point, where the second time point occurs after the first time point and after step (b), wherein the administered compound of formula (I), or a pharmaceutically acceptable salt, prodrug, or isomer thereof, is identified as being effective if the second level is about the same, is decreased as compared to the first level, or is within +/- 20% (e.g., within +/- 15%, within +/- 10%, or within +/- 5%) of the first level. In some embodiments, the methods for determining the efficacy of treatment in a subject having PSP include: (a) determining a first level of neuro-filament light chain (e.g., by performing an immunoassay) in a sample comprising blood or cerebrospinal fluid obtained from the subject at a first time point; (b) administering a therapeutically effective amount of a compound of formula (I), or a pharmaceutically acceptable salt, prodrug, or isomer thereof to the subject after the first time point; and (c) determining a second level of neuro-filament light chain (e.g., by performing an immunoassay) in a sample comprising blood or cerebrospinal fluid obtained from the subject at a second time point, where the second time point occurs after the first time point and after step (b), wherein the administered compound of formula (I), or a pharmaceutically acceptable salt, prodrug, or isomer thereof, is identified as being effective if the second level is about the same, is decreased as compared to the first level, or is within +/- 20% (e.g., within +/- 15%, within +/-10%, or within +/- 5%) of the first level.

Also provided herein are methods of monitoring a subject having PSP receiving a treatment including a compound of formula (I), or a pharmaceutically acceptable salt, prodrug, or isomer thereof, that include: determining two or more levels of a biomarker (e.g., by performing an immunoassay) on different samples comprising blood or cerebrospinal fluid obtained from a subject having PSP (e.g., any of the types of PSP described herein) at different time points. In some embodiments, the biomarker is neurofilament light chain. Some embodiments of these methods can further include performing MRI, CT scan, or PET scan on the subject’s brain.

In some embodiments, methods of determining the efficacy of treatment or the methods of monitoring a subject can further include assessing the severity, duration, and/or frequency of one or more additional symptoms of PSP in the subject (e.g., any of the symptoms of PSP described herein using any of the exemplary techniques or metrics described herein).

Some embodiments of any of the methods described herein can further include administering physical therapy, speech and language therapy, occupational therapy, and/or an antidepressant to the subject,

Also provided herein are methods of treating a subject that include selecting a subject (e.g., any of the subjects described herein) having an elevated level of neurofilament light chain protein in a sample including cerebrospinal fluid, blood, serum, or plasma obtained from the subject, as compared to a reference level of neurofilament light chain protein (e.g., any of the reference levels of neurofilament light chain protein described herein); and administering a pharmaceutical composition including a therapeutically effective amount of a compound of formula (I), or a pharmaceutically acceptable salt, prodrug, or isomer thereof (e.g., using any of the doses or frequencies of administration described herein), to the selected subject.

Also provided are methods of selecting a treatment for a subject (e.g., any of the subjects described herein) that include identifying a subject having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject.

Provided herein are methods of selecting a subject for treatment that include: identifying a subject (e.g., any of the subjects described herein) having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for treatment with a pharmaceutical composition including a therapeutically effective amount of a compound of formula (I), or a pharmaceutically acceptable salt, prodrug, or isomer thereof.

Provided herein are methods of selecting a subject for participation in a clinical trial that include: identifying a subject (e.g., any of the subjects described herein) having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that includes administration of a pharmaceutical composition comprising a therapeutically effective amount of a compound of formula (I), or a pharmaceutically acceptable salt, prodrug, or isomer thereof.

Provided herein are methods of predicting the efficacy of a treatment in a subject (e.g., any of the subjects described herein) that include: determining a level of neurofilament light chain protein level in a sample including cerebrospinal fluid, blood, serum, or plasma obtained from the subject; and determining that a treatment with a pharmaceutical composition including a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein (e.g., any of the exemplary reference levels of neurofilament light chain protein described herein), as compared to a subject not having an elevated level of neurofilament light chain protein in a sample including blood, serum, or plasma as compared to the reference level of neurofilament light chain protein.

Provided herein are methods of determining the efficacy of a treatment in a subject (e.g., any of the subjects described herein) that include: determining a first level of neurofilament light chain protein level in a sample including cerebrospinal fluid, blood, serum, or plasma obtained from the subject at a first time point; determining a second level of neurofilament light chain protein level in a sample including cerebrospinal fluid, blood, serum, or plasma obtained from the subject at a second time point, where the subject received at least one (e.g., at least two, at least three, at least four, at least five, at least six, at least seven, at least eight, at least nine, or at least ten) dose of a pharmaceutical composition including a therapeutically effective amount of a compound of formula (I), or a pharmaceutically acceptable salt, prodrug, or isomer thereof, between the first and the second time points; and identifying the pharmaceutical composition as being effective in a subject having a reduced second level of neurofilament light chain protein as compared to the first level of neurofilament light chain protein.

Provided herein are methods of treating a subject that includes: selecting a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subject.

Provided herein are methods of selecting a treatment for a subject that include identifying a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject.

Also provided herein are methods of selecting a subject for treatment that include: identifying a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject for participation in a clinical trial that include: identifying a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for participation in a clinical trial that includes administration of a pharmaceutical composition including a therapeutically effective amount of a compound of formula (I), or a pharmaceutically acceptable salt, prodrug, or isomer thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the detailed description and figures, and from the claims.

### Neurofilament Light Chain Protein and Methods of Detecting Neurofilament Light Chain Protein

Neurofilaments are type IV intermediate filaments and heteropolymers. Neurofilaments are expressed in the brain and are unique to neuronal cells. Neurofilaments in the central nervous system include neurofilament heavy chain (NFH), neurofilament medium chain (NFM), neurofilament light chain (NFL), and α-internexin. Neurofilaments in the peripheral nervous system comprise NFH, NFM, NFL, and peripherin. Neurofilaments are vital for the maintenance of axon caliber, axon radial growth, and the intracellular transport of electrical impulses along axons (Eyer and Peterson, Neuron 12: 389-405, 1994; Ohara et al., J. Cell Biol. 121: 387-395, 1993; Zhu et al., Exp. Neurol. 148: 299-316, 1997).

Mutations in the NFL gene have been shown to cause Charcot-Marie Tooth disease, demyelinating type 1F (CMT1F), and Charcot-Marie Tooth disease, axonal type 2E (CMT2). Mutations in the NFL gene affect the assembly of neurofilaments in neurons (Sasaki et al., Hum. Mol.Genet. 15: 943-952, 2006; and Yates et al., Eur. J. Cell Biol. 88: 193-202, 2009).

Together with the medium and heavy subunits, neurofilament light chain represents one of the scaffolding proteins of the neuronal cytoskeleton (Teunissen et al., Mult. Scler. 18(5):552-556, 2012) and is released in the extracellular space following axonal damage.

The cDNA sequence and protein sequence of human neurofilament light chain is provided in SEQ ID NO: 1 and SEQ ID NO: 2, respectively. The cDNA sequence and protein sequence of mouse neurofilament light chain is provided in SEQ ID NO: 3 and SEQ ID NO: 4, respectively.

### Human Neurofilament Light Chain cDNA Sequence (SEQ ID NO: 1)

ATGAGTTCCTTCAGCT ACGAGCCGTA CTACTCGACC TCCTACAAGC GGCGCTACGT
GGAGACGCCCCGGGTGCACA TCTCCAGCGT GCGCAGCGGC TACAGCACCG
CACGCTCAGC TTACTCCAGCTACTCGGCGC CGGTGTCTTC CTCGCTGTCC
GTGCGCCGCA GCTACTCCTC CAGCTCTGGATCGTTGATGC CCAGTCTGGA
GAACCTCGAC CTGAGCCAGG TAGCCGCCAT CAGCAACGACCTCAAGTCCA
TCCGCACGCA GGAGAAGGCG CAGCTCCAGG ACCTCAATGA CCGCTTCGCC
AGCTTCATCG AGCGCGTGCA CGAGCTGGAG CAGCAGAACA AGGTCCTGGA
AGCCGAGCTGCTGGTGCTGC GCCAGAAGCA CTCCGAGCCA TCCCGCTTCC
GGGCGCTGTA CGAGCAGGAGATCCGCGACC TGCGCCTGGC GGCGGAAGAT
GCCACCAACG AGAAGCAGGC GCTCCAGGGCGAGCGCGAAG GGCTGGAGGA
GACCCTGCGC AACCTGCAGG CGCGCTATGA AGAGGAGGTGCTGAGCCGCG
AGGACGCCGA GGGCCGGCTG ATGGAAGCGC GCAAAGGCGC CGACGAGGCG
GCGCTCGCTC GCGCCGAGCT CGAGAAGCGC ATCGACAGCT TGATGGACGA
AATCTCTTTTCTGAAGAAAG TGCACGAAGA GGAGATCGCC GAACTGCAGG
CGCAGATCCA GTACGCGCAGATCTCCGTGG AGATGGACGT GACCAAGCCC
GACCTTTCCG CCGCGCTCAA GGACATCCGCGCGCAGTACG AGAAGCTGGC
CGCCAAGAAC ATGCAGAACG CTGAGGAATG GTTCAAGAGCCGCTTCACCG
TGCTGACCGA GAGCGCCGCC AAGAACACCG ACGCCGTGCG CGCCGCCAAG
GACGAGGTGT CCGAGAGCCG TCGTCTGCTC AAGGCCAAGA CCCTGGAAAT
CGAAGCATGCCGGGGCATGA ATGAAGCGCT GGAGAAGCAG CTGCAGGAGC
TGGAGGACAA GCAGAACGCCGACATCAGCG CTATGCAGGA CACGATCAAC
AAATTAGAAA ATGAATTGAG GACCACAAAGAGTGAAATGG CACGATACCT
AAAAGAATAC CAAGACCTCC TCAACGTGAA GATGGCTTTGGATATTGAGA
TTGCAGCTTA CAGGAAACTC TTGGAAGGCG AGGAGACCCG ACTCAGTTTC
ACCAGCGTGG GAAGCATAAC CAGTGGCTAC TCCCAGAGCT CCCAGGTCTT
TGGCCGATCTGCCTACGGCG GTTTACAGAC CAGCTCCTAT CTGATGTCCA
CCCGCTCCTT CCCGTCCTACTACACCAGCC ATGTCCAAGA GGAGCAGATC
GAAGTGGAGG AAACCATTGA GGCTGCCAAGGCTGAGGAAG CCAAGGATGA
GCCCCCCTCT GAAGGAGAAG CCGAGGAGGA GGAGAAGGACAAGGAAGAGG
CCGAGGAAGA GGAGGCAGCT GAAGAGGAAG AAGCTGCCAA GGAAGAGTCT
GAAGAAGCAA AAGAAGAAGA AGAAGGAGGT GAAGGTGAAG AAGGAGAGGA
AACCAAAGAAGCTGAAGAGG AGGAGAAGAA AGTTGAAGGT GCTGGGGAGG
AACAAGCAGC TAAGAAGAAAGATTGA

### Human Neurofilament Light Chain Amino Acid Sequence (SEQ ID NO: 2)

MSSFSYEPYYSTSYKRRYVETPRVHISSVRSGYSTARSAYSSYSAPVSSS
LSVRRSYSSSSGSLMPSLENLDLSQVAAISNDLKSIRTQEKAQLQDLNDR
FASFIERVHELEQQNKVLEAELLVLRQKHSEPSRFRALYEQEIRDLRLAA
EDATNEKQALQGEREGLEETLRNLQARYEEEVLSREDAEGRLMEARKGAD
EAALARAELEKRIDSLMDEISFLKKVHEEEIAELQAQIQYAQISVEMDVT
KPDLSAALKDIRAQYEKLAAKNMQNAEEWFKSRFTVLTESAAKNTDAVRA
AKDEVSESRRLLKAKTLEIEACRGMNEALEKQLQELEDKQNADISAMQDT
INKLENELRTTKSEMARYLKEYQDLLNVKMALDIEIAAYRKLLEGEETRL
SFTSVGSITSGYSQSSQVFGRSAYGGLQTSSYLMSTRSFPSYYTSHVQEE
QIEVEETIEAAKAEEAKDEPPSEGEAEEEEKDKEEAEEEEAAEEEEAAKE
ESEEAKEEEEGGEGEEGEETKEAEEEEKKVEGAGEEQAAKKKD

### Mouse Neurofilament Light Chain cDNA Sequence (SEQ ID NO: 3)

ATGAGTTCGTTCGGCTACGATCCGTACTTTTCGACCTCCTACAAGCGGCG
CTATGTGGAGACGCCCCGGGTGCACATCTCCAGCGTGCGCAGCGGCTACA
GCACGGCGCGCTCCGCGTACTCCAGCTACTCCGCGCCGGTCTCCTCCTCG
CTGTCCGTGCGCCGCAGCTACTCGTCCAGCTCTGGCTCTTTGATGCCCAG
CCTGGAGAATCTCGATCTGAGCCAGGTAGCCGCCATCAGCAACGACCTCA
AGTCTATCCGCACACAAGAGAAGGCACAGCTGCAGGACCTCAACGATCGC
TTCGCCAGCTTCATCGAGCGCGTGCACGAGCTGGAGCAGCAGAACAAGGT
CCTGGAAGCCGAGCTGTTGGTGCTGCGCCAGAAACACTCTGAGCCTTCCC
GCTTCCGCGCCCTGTACGAGCAGGAGATCCGCGATCTGCGGCTGGCAGCG
GAAGACGCCACTAACGAGAAGCAGGCGCTGCAGGGCGAGCGCGAGGGGCT
GGAGGAGACTCTGCGCAACCTGCAGGCTCGCTATGAGGAAGAAGTGCTGA
GCCGCGAGGACGCCGAGGGCCGGCTGATGGAAGCGCGCAAAGGTGCGGAT
GAGGCCGCGCTCGCCCGCGCCGAGCTGGAGAAGCGCATCGACAGCCTGAT
GGACGAGATAGCTTTCCTGAAGAAGGTGCACGAGGAAGAGATCGCCGAGC
TGCAGGCTCAGATCCAGTATGCTCAGATCTCCGTGGAGATGGACGTGTCC
TCCAAGCCCGACCTCTCCGCCGCTCTCAAGGACATCCGCGCTCAGTACGA
GAAGCTGGCCGCCAAGAACATGCAGAACGCCGAAGAGTGGTTCAAGAGCC
GCTTCACCGTGCTAACCGAGAGCGCCGCCAAGAACACCGACGCTGTGCGC
GCTGCCAAGGACGAGGTGTCGGAAAGCCGCCGCCTGCTCAAGGCTAAGAC
CCTGGAGATCGAAGCCTGCCGGGGTATGAACGAAGCTCTGGAGAAGCAGC
TGCAGGAGCTAGAGGACAAGCAGAATGCAGACATTAGCGCCATGCAGGAC
ACAATCAACAAACTGGAGAATGAGCTGAGAAGCACGAAGAGCGAGATGGC
CAGGTACCTGAAGGAGTACCAGGACCTCCTCAATGTCAAGATGGCCTTGG
ACATCGAGATTGCAGCTTACAGAAAACTCTTGGAAGGCGAAGAGACCAGG
CTCAGTTTCACCAGCGTGGGTAGCATAACCAGCGGCTACTCTCAGAGCTC
GCAGGTCTTCGGCCGTTCTGCTTACAGTGGCTTGCAGAGCAGCTCCTACT
TGATGTCTGCTCGCTCTTTCCCAGCCTACTATACCAGCCACGTCCAGGAA
GAGCAGACAGAGGTCGAGGAGACCATTGAGGCTACGAAAGCTGAGGAGGC
CAAGGATGAGCCCCCCTCTGAAGGAGAAGCAGAAGAGGAGGAGAAGGAGA
AAGAGGAGGGAGAGGAAGAGGAAGGCGCTGAGGAGGAAGAAGCTGCCAAG
GATGAGTCTGAAGACACAAAAGAAGAAGAAGAAGGTGGTGAGGGTGAGGA
GGAAGACACCAAAGAATCTGAAGAGGAAGAGAAGAAAGAGGAGAGTGCTG
GAGAGGAGCAGGTGGCTAAGAAGAAAGATTGA

### Mouse Neurofilament Light Chain Amino Acid Sequence (SEQ ID NO: 4)

MSSFGYDPYFSTSYKRRYVETPRVHISSVRSGYSTARSAYSSYSAPVSSS
LSVRRSYSSSSGSLMPSLENLDLSQVAAISNDLKSIRTQEKAQLQDLNDR
FASFIERVHELEQQNKVLEAELLVLRQKHSEPSRFRALYEQEIRDLRLAA
EDATNEKQALQGEREGLEETLRNLQARYEEEVLSREDAEGRLMEARKGAD
EAALARAELEKRIDSLMDEIAFLKKVHEEEIAELQAQIQYAQISVEMDVS
SKPDLSAALKDIRAQYEKLAAKNMQNAEEWFKSRFTVLTESAAKNTDAVR
AAKDEVSESRRLLKAKTLEIEACRGMNEALEKQLQELEDKQNADISAMQD
TINKLENELRSTKSEMARYLKEYQDLLNVKMALDIEIAAYRKLLEGEETR
LSFTSVGSITSGYSQSSQVFGRSAYSGLQSSSYLMSARSFPAYYTSHVQE
EQTEVEETIEATKAEEAKDEPPSEGEAEEEEKEKEEGEEEEGAEEEEAAK
DESEDTKEEEEGGEGEEEDTKESEEEEKKEESAGEEQVAKKKD

Some embodiments of any of the methods described herein can include a step of performing an assay to determine a level or levels (e.g., first and second level) of neurofilament light chain protein in a sample or samples (e.g., samples obtained from the subject at a first and a second time point). Non-limiting assays that may be used to detect a level or levels of neurofilament light chain are described herein. Additional assays that may be used to detect a level or levels of neurofilament light chain are known in the art.

A commercially-available enzyme-linked immunosorbent assay (UmanDiagnostics) can be used to measure a level or levels of neurofilament light chain protein in a sample or samples including cerebrospinal fluid, blood, serum, or plasma from a subject. An electrochemiluminescence (ECL)-based assay can also be used to detect a level or levels of neurofilament light chain. See, e.g., Lycke et al., J. Neurol. Neurosurg. Psychiatry 64(3):402-404, 1998; Teunissen et al., Neurology 72(15): 1322-1329, 2009; Disanto et al., J. Neurol. Neurosurg. Psychiatry 87(2):126-129, 2015; and Kuhle et al., Mult. Scler. 22(12): 1550-1559, 2016). Another assay that can be performed to detect a level or levels of neurofilament light chain protein is a single-molecule array (Simoa) assay, which is described in detail in Kuhle et al., Clin. Chem. Lab Med. 54(10): 1655-166, 2016; and Gisslen et al., EBioMedicine 3:135-140, 2016). A Simoa assay for neurofilament light chain is commercially available from Quanterix (NF-LIGHT®). The NF-LIGHT® Quanterix Simoa assay has been used to detect levels of neurofilament light chain protein in samples including cerebrospinal fluid, blood, serum, or plasma from human subjects. The antibodies used in the NF-LIGHT® Quanteriz Simoa assay (obtained from Uman Diagnostics, Umea, Sweden) show cross-reactivity with human, mouse, bovine, and macaque neurofilament light chain proteins. The NF-LIGHT® Quanterix Simoa assay is a digital immunoassay.

Additional non-limiting assays that can be used to detect a level of neurofilament light chain protein in a sample include: enzyme-linked immunosorbent assay (ELISA), sensitive sandwich ELISA assay, electrochemiluminescence (ECL)-based assay, mass spectrometry (MS), western blotting, fluorescence-activated cell sorting (FACS), immunohistochemistry.

Other assays that can be used to detect a level or levels of neurofilament light chain in a sample obtained from a subject (e.g., any of the exemplary samples described herein) include immunoassays (e.g., enzyme-linked immunosorbent assays, sandwich enzyme-linked immunosorbent assays, and immunoprecipitation) and proteomic techniques.

### Detecting Neurofilament Light Chain Protein

### Assay for Detecting Neurofilament Light Chain (NFL) in Serum, Plasma, or Blood Samples

Single-molecule array (Simoa) assay is a highly sensitive assay that allows accurate quantification of low neurofilament light chain concentrations (Disanto et al., Ann. Neurol. 81(6):857-870, 2017; Rohrer et al., Neurology 87(13):1329-1336, 2016; and Novakova et al., Neurology 89(22): 2230-2237, 2017).

Capture monoclonal antibody (mAB) 47: 3 is buffer exchanged and diluted to 0.3 mg/mL 4 x 10⁶ paramagnetic beads (Quanterix Corporation) are buffer exchanged and activated using 0.5 mg/mL 1-ethyl-3-(3-dimetylaminopropyl) carbodiimide (Quanterix), followed by a 30-minute incubation at room temperature (HulaMixer, Thermo Fischer Scientific). The diluted capture mAB 47:3 is conjugated with the washed and activated paramagnetic beads for a 2-hour incubation at room temperature on a mixer. After the incubation, the beads are washed three times using a magnetic separator and blocked. Next, the conjugated beads are suspended and stored at 4° C.

The Simoa assay is run on a Simoa HD-1 instrument (Quanterix) using a two-step Assay Neat 20 protocol. Briefly, 100 µL of calibrator/sample in Tris-buffered saline (TBS), 0.1% Tween 20, 1% milk powder, 400 µg/mL Heteroblock (Omega Biologicals), 25 µL conjugated beads in TBS, 0.1% Tween 20, 1% milk powder, 300 µg/mL Heteroblock, and 20 µL of mAB 2:1 (0.1 µg/mL in TBS, 0.1% Tween 20, 1% milk powder, 300 µg/mL Heteroblock) are incubated for 35 minutes 15 seconds (47 cadences with 1 cadence is 45 seconds). Next, the mixture is washed. Then, 100 µL, of streptavidin-conjugated β-galactosidase (150 pM; Quanterix) is added, followed by a 5 minutes 15 second (7-cadence) incubation and wash. The mixture will then be placed on a Simoa HD-1 instrument. Prior to reading, 25 µL Resorufin β-D-galactopyranoside (Quanterix) is added. The calibrator curve is constructed using the standard from the NFL ELISA (NF-light, UmanDiagnostics). Samples and calibrators are measured in duplicates.

NFL levels are log-transformed to meet the normal assumption. The distribution of sNfl in healthy control is modeled by means of Generalized Additive Models for Location, Scale, and Shape (GAMLSS) using a Box-Cox t distribution according to Rigby and Stasinopoulous,Stat Med 23: 3053-3076, 2004, and cubic splines and percentile curves are obtained.

To quantify, intra- and interassay variability, bootstrapping is applied by drawing 100 random samples from the healthy controls.

Linear regression models are used to investigate the associations with log sNFL. Linear generalized estimating equation (GEE) models are similarly used to investigate associations with log sNFL.

### Blood NFL Measurements by Ultrasensitive Simoa Assay

Serum Nfl levels are investigated in longitudinal blood samples.

A commercially available ELISA (UmanDiagnostics NF-light® assay) uses two highly specific, non-competing monoclonal antibodies (47:3 and 2:1) to quantify soluble NFL in cerebrospinal fluid (CSF) samples. Using this assay to analyze CSF samples, it was found that NFL is more sensitive than NFH to distinguish patients in different stages of MS versus controls (Kuhle et al., Mult Scler 19:1597-1603, 2013). Together with UmanDiagnostics a highly sensitive electrochemiluminescence (ECL)-based NfL assay was developed suitable for the quantification of NFL in serum at concentrations relevant to clinical settings (Gaiottino et al., PLoS One 2013;8(9):e75091, 2013; and Disanto et al., J Neurol Neurosurg Psychiatry 87(2): 126-129, 2015). The HD-1 analyzer relies on single-molecule arrays (Simoa) and the simultaneous counting of singulated capture microbeads (Rissin et al., Nat Biotechnol 28:595-599, 2010). The instrument integrates established paramagnetic microbead-based reagent robotics with a novel imaging module that digitizes the immunoassay with an array consumable and optical system at a cost similar to that of conventional immunoassay platforms (Wilson et al., JLab Autom 21(4): 533-547, 2015). The system can accommodate user-developed custom reagents and assay protocols, as well as a menu of pre-validated assay kits. Across a range of immunoassays covering several disease areas, the fully automated Simoa assays exhibited ≥4 logs of measurement range, single digit CVs, and sensitivities in the femtogram per milliliter range (Wilson et al., JLab Autom 21(4): 533-547, 2015). Thus, the Simoa HD-1 Analyzer is approximately 3 logs more sensitive than conventional fluorescence, chemiluminescence, and ECL immunoassay instrumentation.

### Reference Levels

In some embodiments of any of the methods described herein, the reference level can be a level of neurofilament light chain protein detected in a similar sample obtained from a subject (e.g., a subject who is between 18 to 70 years of age), that has not been diagnosed or identified as having a neurological disorder (e.g. MS) or neural tissue damage, and does not have a family history of a neurological disorder (e.g., MS) or neural tissue damage. In some embodiments, a reference level can be threshold level of neurofilament light chain protein.

A reference level of neurofilament light chain protein (e.g., for samples including serum) is about 10 pg/mL to about 35 pg/mL (e.g., about 10 pg/mL to about 30 pg/mL, about 10 pg/mL to about 25 pg/mL, about 10 pg/mL to about 20 pg/mL, 10 pg/mL to about 15 pg/mL, 15 pg/mL to about 35 pg/mL about 15 pg/mL to about 30 pg/mL, about 15 pg/mL to about 25 pg/mL, about 15 pg/mL to about 20 pg/mL, about 20 pg/mL to about 35 pg/mL, about 20 pg/mL to about 30 pg/mL, about 20 pg/mL to about 25 pg/mL, about 25 pg/mL to about 35 pg/mL, or about 25 pg/mL or about 30 pg/mL).

In some embodiments, a reference level of neurofilament light chain protein can be a percentile value (e.g., mean value, 99% percentile, 95% percentile, 90% percentile, 85% percentile, 80% percentile, 75% percentile, 70% percentile, 65% percentile, 60% percentile, 55% percentile, or 50% percentile) of the levels of neurofilament light chain protein detected in similar samples in a population of healthy subjects (e.g., subjects that are not diagnosed or identified as having a disease (e.g., any of the neurological disorders described herein or neural tissue damage), do not present with a symptom of a disorder or disease (e.g., a neurological disease or disorder), and are not considered to have an elevated risk of developing a neurological disease or disorder).

In some embodiments, a reference level can be the level of neurofilament light chain detected in a similar sample obtained from the subject at an earlier time point.

### CHS-131 (INT-131)

CHS-131 is an exemplary compound of formula (I).

In one aspect, CHS-131 reduces neural inflammation in a subject having been diagnosed as having a neurological disorder (e.g., multiple sclerosis (MS), relapsing-remitting MS (RRMS), or Alzheimer’s disease) or neural tissue damage, or a subject that does not present with a symptom of a neurological disorder (e.g., multiple sclerosis (MS), relapsing-remitting MS (RRMS), or Alzheimer’s disease) or neural tissue damage. In some embodiments, a subject can be identified or diagnosed as having an early stage of a neurological disorder or an early stage or mild neural tissue damage.

In some embodiments, administration of CHS-131 reduces atrophy or degeneration of the brain in the subject (e.g., any of the subjects described herein). In some embodiments, administration of CHS-131 reduces atrophy or degradation of the substantia nigra, globus palladus, subthalamic nucleus and/or cerebellum in the subject (e.g., any of the subjects described herein). In some embodiments, administration of CHS-131 reduces cortical atrophy in the brain of the subject (e.g., any of the subjects described herein).

In one aspect, administration of CHS-131 decreases the risk of developing a comorbidity (e.g., cardiovascular disease, type 2 diabetes mellitus) in the subject (e.g., any of the subjects described herein).

In one embodiment, CHS-131 is as effective, or more effective, treating a neurological disorder or neural tissue damage than other therapies. These therapies include therapies approved for treating a neurological disorder or neural tissue damage (e.g., any of the neurological disorders described herein) and those in development for treating a neurological disorder or neural tissue damage (e.g., any of the neurological disorder described herein).

In some embodiments of any of the methods described herein, a pharmaceutical composition including a therapeutically effective amount of formula (I), or a pharmaceutically acceptable salt, prodrug, or isomer thereof, can be co-administered to the subject with one or more additional treatments (e.g., any of the other exemplary treatments or therapies described herein).

### Additional Methods of Treating

Provided herein are methods of treating a subject that include: selecting a subject (e.g., any of the exemplary subjects described herein) having an elevated level of neurofilament light chain protein in a sample including cerebrospinal fluid, blood, serum, or plasma obtained from the subject, as compared to a reference level of neurofilament light chain protein (e.g., any of the exemplary reference levels of neurofilament light chain protein described herein or known in the art); and administering a pharmaceutical composition including a therapeutically effective amount of a compound of formula (I), or a pharmaceutically acceptable salt, prodrug, or isomer thereof, to the selected subject (e.g., e.g., CHS-131) using any of the doses or frequencies of administration described herein).

Also provided are methods of treating a subject (e.g., any of the subjects described herein) that include: selecting a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and administering a pharmaceutical composition including a therapeutically effective amount of a compound of formula (I), or a pharmaceutically acceptable salt, prodrug, or isomer thereof, to the selected subject (e.g., CHS-131, e.g., using any of the doses or frequencies of administration described herein).

### Methods of Selecting a Treatment for a Subject

Provided herein are methods of selecting a treatment for a subject (e.g., any of the subjects described herein) that include: identifying a subject having an elevated level of neurofilament light chain protein in a sample comprising cerebrospinal fluid, blood, serum, or plasma obtained from the subject, as compared to a reference level of neurofilament light chain protein (e.g., any of the exemplary reference levels of neurofilament light chain protein described herein); and selecting a pharmaceutical composition including a therapeutically effective amount of a compound of formula (I), or a pharmaceutically acceptable salt, prodrug, or isomer thereof, for the identified subject.

Also provided are methods of selecting a treatment for a subject (e.g., any of the subjects described herein) that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting a pharmaceutical composition including a therapeutically effective amount of a compound of formula (I), or a pharmaceutically acceptable salt, prodrug, or isomer thereof, for the identified subject. In any of the methods that include detecting a level of neurofilament light chain protein in a first and a second sample, one skilled in the art will appreciate that, e.g., the samples should be similar (e.g., both samples are serum samples, both samples are blood samples, both samples are plasma samples, or both samples are cerebrospinal fluid samples).

Some embodiments of these methods further include administering one or more doses (e.g., at least two, at least five, or at least ten doses) of the selected pharmaceutical composition to the identified subject. Some embodiments of these methods further include recording the selected pharmaceutical composition in the identified subject’s clinical records.

Also provided herein are methods of selecting a subject for treatment that include: identifying a subject having an elevated level of neurofilament light chain protein in a sample comprising cerebrospinal fluid, blood, serum, or plasma obtained from the subject, as compared to a reference level of neurofilament light chain protein; and selecting the identified subject for treatment with a pharmaceutical composition comprising a therapeutically effective amount of a compound of formula (I), or a pharmaceutically acceptable salt, prodrug, or isomer thereof.

Also provided are methods of selecting a subject (e.g., any of the subjects described herein) for treatment that include: identifying a subject having an elevated second level of neurofilament light chain protein in a sample including cerebrospinal fluid, blood, serum, or plasma obtained from the subject at a second time point, as compared to a first level of neurofilament light chain protein in a sample including cerebrospinal fluid, blood, serum, or plasma obtained from the subject at a first time point; and selecting the identified subject for treatment with a pharmaceutical composition including a therapeutically effective amount of a compound of formula (I), or a pharmaceutically acceptable salt, prodrug, or isomer thereof.

Also provided herein are methods of selecting a subject (e.g., any of the subjects described herein) for participation in a clinical trial that include: identifying a subject having an elevated level of neurofilament light chain protein in a sample comprising cerebrospinal fluid, blood, serum, or plasma obtained from the subject, as compared to a reference level of neurofilament light chain protein (e.g., any of the exemplary reference levels of neurofilament light chain protein described herein); and selecting the identified subject for participation in a clinical trial that comprises administration of a pharmaceutical composition comprising a therapeutically effective amount of a compound of formula (I), or a pharmaceutically acceptable salt, prodrug, or isomer thereof (e.g., CHS-131, e.g., using any of the doses or frequencies of administration described herein).

Also provided are methods of selecting a subject for participation in a clinical trial that include: identifying a subject (e.g., any of the subjects described herein) having an elevated second level of neurofilament light chain protein in a sample including cerebrospinal fluid, blood, serum, or plasma obtained from the subject at a second time point, as compared to a first level of neurofilament light chain protein in a sample comprising cerebrospinal fluid, blood, serum, or plasma obtained from the subject at a first time point; and selecting the identified subject for participation in a clinical trial that comprises administration of a pharmaceutical composition comprising a therapeutically effective amount of a compound of formula (I), or a pharmaceutically acceptable salt, prodrug, or isomer thereof (e.g., CHS-131, e.g., using any of the doses or frequencies of administration described herein).

### Methods of Predicting

Provided herein are methods of predicting the efficacy of a treatment in a subject that include: determining a level of neurofilament light chain protein level in a sample comprising cerebrospinal fluid, blood, serum, or plasma obtained from the subject; and determining that a treatment with a pharmaceutical composition comprising a therapeutically effective amount of a compound of formula (I), or a pharmaceutically acceptable salt, prodrug, or isomer thereof, is more likely to be effective in a subject having an elevated level of neurofilament light chain protein in the sample as compared to a reference level of neurofilament light chain protein, as compared to a subject not having an elevated level of neurofilament light chain protein in a sample comprising blood, serum, or plasma as compared to the reference level of neurofilament light chain protein.

In some aspects, the methods can include obtaining from the subject a second sample comprising cerebrospinal fluid, blood, serum, or plasma at a second time point and repeating the determining step.

### Compositions and Kits

Also provided herein are compositions (e.g., pharmaceutical compositions) that include at least one compound of formula (I) of any of the compounds described herein and instructions for performing any of the methods described herein. In some embodiments, the compositions (e.g., pharmaceutical compositions) can be disposed in a sterile vial or a pre-loaded syringe.

In some embodiments, the compositions (e.g., pharmaceutical compositions) are formulated for different routes of administration (e.g., intracranial, intravenous, subcutaneous, or intramuscular). In some embodiments, the compositions (e.g., pharmaceutical compositions) can include a pharmaceutically acceptable salt (e.g., phosphate buffered saline). In some embodiments, the compositions (e.g., pharmaceutical compositions) can include a prodrug, or an isomer thereof. Single or multiple administrations of any of the pharmaceutical compositions described herein can be given to a subject depending on, for example: the dosage and frequency as required and tolerated by the patient. A dosage of the pharmaceutical composition should provide a sufficient quantity of the compound of formula (I) a pharmaceutically acceptable salt, prodrug, or an isomer thereof to effectively treat or ameliorate conditions, diseases or symptoms.

Also provided herein are kits containing one or more (e.g., at least 2, 3, 4, 5, 6, 7, 8, 9, 10, 12, 14, 16, 18, 20) of any of the pharmaceutical compositions described herein that include a therapeutically effective amount of a compound of formula (I), or a pharmaceutically acceptable salt, prodrug, or isomer thereof. In some embodiments, the kits can include instructions for performing any of the methods described herein. In some embodiments, the kits can include at least one dose of any of the compositions (e.g., pharmaceutical compositions) described herein. In some embodiments, the kits can provide a syringe for administering any of the pharmaceutical compositions described herein. The kits described herein are not so limited; other variations will be apparent to one of ordinary skill in the art.

The invention is further described in the following examples, which do not limit the scope of the invention described in the claims.

### EXAMPLES

### Example 1: INT131 is a Potent Upregulator of Adiponectin in Patients With Reduced Adiponectin Levels

### Method

A randomized, double-blind, placebo-controlled, 24-week study was conducted in which adiponectin levels were measured. The study had a 2-week lead-in period, a 24-week double-blind treatment period and a 2-week follow up period. 367 subjects with type 2 diabetes (TD2)—a disease in which patient adiponectin levels are reduced—were randomly assigned to receive either 0.5, 1, 2 or 3 milligrams (“mg”) of INT131 besylate, 45 mg of pioglitazone or placebo daily for 24 weeks. To measure adiponectin levels blood was drawn at Weeks 0, 2, 6, 12 and 24.

The results of this study demonstrated that 1, 2, and 3 mg doses of INT131 caused a statistically significant reduction of HbA_{1c} levels as compared to placebo. Further, the study demonstrated that the 2 and 3 mg doses of INT131 reduced HbA_{1c} levels at least as well as 45 mg of pioglitazone, which is an FDA approved treatment for TD2. See, DePaoli, et al. Diabetes Care 2014;37:1918-1923. Thus, 2 and 3 mg doses of INT131 would be effective in treating TD2.

### Adiponectin Results

At baseline (Week 0) mean adiponectin levels were 1.94 micrograms per milliliter (“µg/mL”). The mean adiponectin levels at baseline and Week 24, and the mean change in adiponectin levels from baseline (Week 0) to Week 24 are disclosed in Table 1, below. The standard deviation for samples tested in each group is listed in (parenthesis). Mean baseline adiponectin values were similar for the treatment groups.

TABLE 1

Changes in Adiponectin Serum Levels

| Mean Adiponectin (µg/mL) | Placebo | 0.5 mg INT131 | 1 mg INT131 | 2 mg INT131 | 3 mg INT131 | 45 mg Pioglitazone |
|---|---|---|---|---|---|---|
| n | 56 | 56 | 59 | 60 | 60 | 57 |
| Week 0 | 1.85 (1.153) | 1.73 (1.190) | 1.87 (1.217) | 1.87 (1.098) | 2.00 (1.215) | 2.32 (2.185) |
| Week 24 | 1.9 (1.510) | 2.28 (1.540) | 3.15 (2.533) | 5.14 (3.650) | 5.83 (4.826) | 5.28 (3.222) |
| Mean Change | 0.05 (0.680) | 0.56 (0.906) | 1.28 (1.882) | 3.27 (3.002) | 3.83 (4.313) | 2.96 (2.618) |

The treatment comparisons of 1 mg, 2 mg, and 3 mg doses of INT131 with placebo were statistically significant (p≤0.0109). This demonstrates that treatment with INT131 resulted in a statistically significant increase in adiponectin levels in patients suffering from a disease in which adiponectin levels are reduced (e.g. TD2). Thus, INT131 is therapeutically effective in treating patients with diseases (e.g. progressive supranuclear palsy) in which increasing lowered adiponectin levels may be beneficial.

Additionally, the treatment comparisons of 0.5 mg, 1 mg, and 3 mg doses of INT131 with pioglitazone 45 mg were statistically significant (p≤0.0408). Thus, the dose dependent increase of adiponectin levels by INT131 is independent from the increase resulting from pioglitazone.

### Conclusions

The effect of treatment on serum adiponectin was assessed, enabling a more direct comparison of the relative potencies of INT131 and pioglitazone 45 mg as selective PPARy modulators. The mean change in adiponectin from baseline to Week 24 with LOCF (last observation carried forward) was 0.05 µg/mL for the placebo group, 0.56 µg/mL for the INT131 0.5 mg group, 1.28 µg/mL for the INT131 1 mg group, 3.27 µg/mL for the 2 mg group, 3.83 µg/mL for the INT131 3 mg group, and 2.96 µg/mL for the pioglitazone 45 mg group. Therefore, in a manner quantitatively different from the effects on HbA_{1c}, where the INT131 dose roughly equivalent to pioglitazone 45 mg is between 2 mg and 3 mg, a dose of INT131 between 1 mg and 2 mg was equivalent to pioglitazone 45 mg for increasing adiponectin levels.

Surprisingly, administration of INT131 at either 2 or 3 mg resulted in a greater upregulation of serum adiponectin levels than did administration of at least 22 times the amount of pioglitazone. Small amounts of INT131 are at least as efficacious in treating diseases in which adiponectin levels are reduced as are other drugs which also increase adiponectin levels. Since INT131 crosses the blood brain barrier more readily than other PPARγ agonists, less INT131 is required to achieve the same increase adiponectin, and INT131 has fewer side effects than other PPARy agonists, INT131 is a superior treatment for neurological diseases.

Administration of 1, 2, or 3 mg of INT131 treats patients suffering from diseases in which prevention or inhibition of adiponectin level decreases, or increasing adiponectin levels, may be beneficial (e.g. progressive supranuclear palsy).

### Example 2: INT131 Is a Potent Upregulator of Adiponectin in Healthy Subjects

### Method

A study was conducted to determine the effect of INT131 on serum adiponectin levels. Thirty healthy subjects were randomly selected to receive either placebo, 0.1 mg INT131, 1 mg INT131 or 4 mg INT131 daily for 14 days. To measure adiponectin levels blood was drawn at Days 1, 4, 8 and 14.

### Results

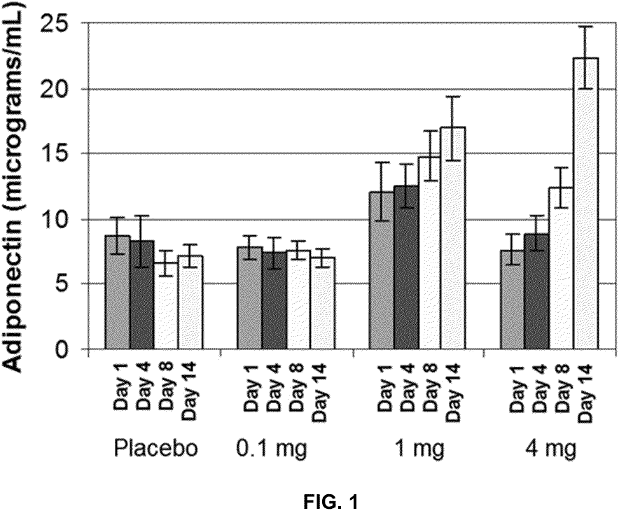

From Day 1 to Day 14 administration of placebo and 0.1 mg INT131 resulted in no significant change in serum adiponectin levels and further administration of 0.1 mg INT131 resulted in no significant change in adiponectin levels over placebo. See FIG. 1. However, administration of 1 mg or 4 mg INT131 resulted in a significant change in serum adiponectin levels over placebo and a significant change from Day 1 to Day 14. Thus, administration of INT131 is capable of upregulating adiponectin in healthy individuals (i.e. subjects without reduced adiponectin levels as a result of a disease or disorder).

Since INT131 upregulates adiponectin, even if a subject’s adiponectin level has not been reduced by disease or disorder, upregulation of adiponectin in a subject with progressive supranuclear palsy may be beneficial in delaying progression of signs and symptoms of the disease.

### Example 3: INT131 Is Neuroprotective in Neurodegenerative Disease and Is Useful in Treating PSP

### Methods

227 treatment-naive adult subjects (mean age 31 [range 18-50]; 65% female) were randomized into one of three arms: oral CHS-131 at 3 mg (n=76); oral CHS-131 at 1 mg (n=76); placebo (n=75), at 21 sites in Russia. 97% completed Part 1. Inclusion criteria: Relapsing Remitting Multiple Sclerosis (RRMS) for ≤3 years, ≥1 gadolinium-positive lesion within 1-year of enrollment, and an EDSS ≤ 6 at screening. The patients underwent monthly MRI examinations with contrast to identify new inflammatory lesions. All MRIs were read blinded at the Buffalo Neuroimaging Center (Buffalo, NY). In addition to CE lesions the images were scored for cumulative new/enlarged T2 lesions, and serial cortical and whole brain volumetric analysis.

### Results

CHS-131 treatment resulted in a dose-dependent reduction in cumulative CE lesions over 6 months (complete case analysis). 3 mg daily reduced CE lesion burden by 52% (4.2 lesions [LSMean 3.10]) vs. placebo (7.8 [LSMean 6.49]) (p=0.003). 1 mg treatment was less robust- 21% reduction vs. placebo (7.6 [LSMean 5.15] (p=ns). New/enlarged T2 lesions were reduced by 30% (3 mg)(p=0.0767) and 14% (1 mg)(p=ns) compared to placebo.

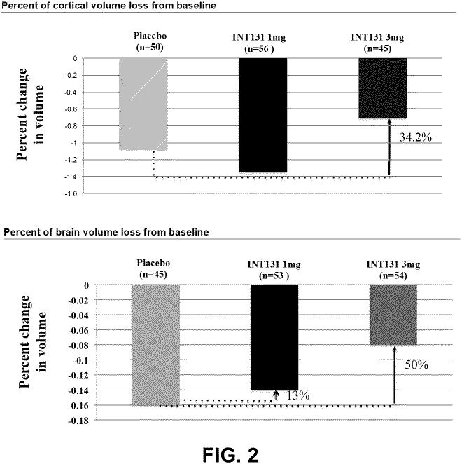

In addition to the dose-dependent anti-neuroinflammatory effects, CHS-131 treatment appeared to attenuate gray matter volume loss at 6-months. Compared to placebo, there was 34.2% less cortical volume loss and 50% less whole brain volume loss in the 3 mg cohort. Volume losses in the 1 mg treatment group were similar to placebo at 6 months. See, FIG. 2.

These data demonstrate that CHS-131 is neuroprotective. Therefore, administration of CHS-131 to a subject with progressive supranuclear palsy may be beneficial in delaying progression of signs and symptoms of the disease.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-48. (canceled)

49. A method of treating a subject, the method comprising:
selecting a subject having an elevated level of neurofilament light chain protein in a sample comprising cerebrospinal fluid, blood, serum, or plasma obtained from the subject, as compared to a reference level of neurofilament light chain protein; and
administering a pharmaceutical composition comprising a therapeutically effective amount of a compound of formula (I), 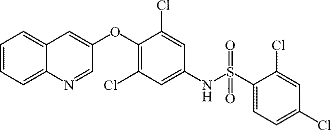 [SMILES: O=S(=O)(Nc1cc(Cl)c(Oc2cnc3ccccc3c2)c(Cl)c1)c1ccc(Cl)cc1Cl]
or a pharmaceutically acceptable salt, thereof, to the selected subject.

50-53. (canceled)

54. The method of claim 49, wherein the subject has not been diagnosed with progressive supranuclear palsy.

55. The method of claim 49, wherein the subject does not present with a symptom of progressive supranuclear palsy.

56. The method of claim 49, wherein the subject has been diagnosed as having progressive supranuclear palsy.

57. The method of claim 49, wherein the method further comprises performing an assay to determine the level of neurofilament light chain protein in the sample obtained from the subject.

58. The method of claim 49, wherein the assay is a single-molecule array assay.

59. The method of claim 49, wherein the method further comprises administering the selected treatment to the identified subject.

60. The method of claim 49, wherein the subject has been previously administered a different pharmaceutical composition and the different pharmaceutical composition was determined not to be therapeutically effective.

61-74. (canceled)

75. The method of claim 49, wherein the compound of formula (I) is in the form of a besylate salt.

76. The method of claim 49, wherein the therapeutically effective amount is from about 0.1 to about 15 milligrams.

77. The method of claim 76, wherein therapeutically effective amount is from about 1 to about 10 milligrams.

78. The method of claim 77, wherein the therapeutically effective amount is from about 2 to about 6 milligrams.

79. The method of claim 78, wherein the therapeutically effective amount is about 3 milligrams.

80. The method of claim 49, wherein the pharmaceutical composition is administered to the subject twice a day, daily, every other day, three times a week, twice a week, weekly, every other week, twice a month, or monthly.

81. The method of claim 80, wherein the pharmaceutical composition is administered to the subject daily.

82. The method of claim 49, wherein the pharmaceutical composition is administered to the subject daily and the therapeutically effective amount of the compound is from about 1 to about 10 milligrams.

83. The method of claim 49, wherein the pharmaceutical composition is administered to the subject daily and the therapeutically effective amount of the compound is from about 2 to about 6 milligrams.

84. The method of claim 49, wherein the pharmaceutical composition is administered to the subject daily and the therapeutically effective amount of the compound is about 1 milligram.

85. The method of claim 49, wherein the pharmaceutical composition is administered to the subject daily and the therapeutically effective amount of the compound is about 2 milligrams.

86. The method of claim 49, wherein the pharmaceutical composition is administered to the subject daily and the therapeutically effective amount of the compound is about 3 milligrams.


SEQUENCE LISTING (4 sequences)

SEQ ID NO: 1 - DNA, 1632 bp, Human
atgagttccttcagctacgagccgtactactcgacctcctacaagcggcgctacgtggagacgccccgggtgcacatctccagcgtgcgcagcggctacagcaccgcacgctcagcttactccagctactcggcgccggtgtcttcctcgctgtccgtgcgccgcagctactcctccagctctggatcgttgatgcccagtctggagaacctcgacctgagccaggtagccgccatcagcaacgacctcaagtccatccgcacgcaggagaaggcgcagctccaggacctcaatgaccgcttcgccagcttcatcgagcgcgtgcacgagctggagcagcagaacaaggtcctggaagccgagctgctggtgctgcgccagaagcactccgagccatcccgcttccgggcgctgtacgagcaggagatccgcgacctgcgcctggcggcggaagatgccaccaacgagaagcaggcgctccagggcgagcgcgaagggctggaggagaccctgcgcaacctgcaggcgcgctatgaagaggaggtgctgagccgcgaggacgccgagggccggctgatggaagcgcgcaaaggcgccgacgaggcggcgctcgctcgcgccgagctcgagaagcgcatcgacagcttgatggacgaaatctcttttctgaagaaagtgcacgaagaggagatcgccgaactgcaggcgcagatccagtacgcgcagatctccgtggagatggacgtgaccaagcccgacctttccgccgcgctcaaggacatccgcgcgcagtacgagaagctggccgccaagaacatgcagaacgctgaggaatggttcaagagccgcttcaccgtgctgaccgagagcgccgccaagaacaccgacgccgtgcgcgccgccaaggacgaggtgtccgagagccgtcgtctgctcaaggccaagaccctggaaatcgaagcatgccggggcatgaatgaagcgctggagaagcagctgcaggagctggaggacaagcagaacgccgacatcagcgctatgcaggacacgatcaacaaattagaaaatgaattgaggaccacaaagagtgaaatggcacgatacctaaaagaataccaagacctcctcaacgtgaagatggctttggatattgagattgcagcttacaggaaactcttggaaggcgaggagacccgactcagtttcaccagcgtgggaagcataaccagtggctactcccagagctcccaggtctttggccgatctgcctacggcggtttacagaccagctcctatctgatgtccacccgctccttcccgtcctactacaccagccatgtccaagaggagcagatcgaagtggaggaaaccattgaggctgccaaggctgaggaagccaaggatgagcccccctctgaaggagaagccgaggaggaggagaaggacaaggaagaggccgaggaagaggaggcagctgaagaggaagaagctgccaaggaagagtctgaagaagcaaaagaagaagaagaaggaggtgaaggtgaagaaggagaggaaaccaaagaagctgaagaggaggagaagaaagttgaaggtgctggggaggaacaagcagctaagaagaaagattga

SEQ ID NO: 2 - protein, 543 aa, Human
MSSFSYEPYYSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEISFLKKVHEEEIAELQAQIQYAQISVEMDVTKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRTTKSEMARYLKEYQDLLNVKMALDIEIAAYRKLLEGEETRLSFTSVGSITSGYSQSSQVFGRSAYGGLQTSSYLMSTRSFPSYYTSHVQEEQIEVEETIEAAKAEEAKDEPPSEGEAEEEEKDKEEAEEEEAAEEEEAAKEESEEAKEEEEGGEGEEGEETKEAEEEEKKVEGAGEEQAAKKKD

SEQ ID NO: 3 - DNA, 1632 bp, Human
atgagttcgttcggctacgatccgtacttttcgacctcctacaagcggcgctatgtggagacgccccgggtgcacatctccagcgtgcgcagcggctacagcacggcgcgctccgcgtactccagctactccgcgccggtctcctcctcgctgtccgtgcgccgcagctactcgtccagctctggctctttgatgcccagcctggagaatctcgatctgagccaggtagccgccatcagcaacgacctcaagtctatccgcacacaagagaaggcacagctgcaggacctcaacgatcgcttcgccagcttcatcgagcgcgtgcacgagctggagcagcagaacaaggtcctggaagccgagctgttggtgctgcgccagaaacactctgagccttcccgcttccgcgccctgtacgagcaggagatccgcgatctgcggctggcagcggaagacgccactaacgagaagcaggcgctgcagggcgagcgcgaggggctggaggagactctgcgcaacctgcaggctcgctatgaggaagaagtgctgagccgcgaggacgccgagggccggctgatggaagcgcgcaaaggtgcggatgaggccgcgctcgcccgcgccgagctggagaagcgcatcgacagcctgatggacgagatagctttcctgaagaaggtgcacgaggaagagatcgccgagctgcaggctcagatccagtatgctcagatctccgtggagatggacgtgtcctccaagcccgacctctccgccgctctcaaggacatccgcgctcagtacgagaagctggccgccaagaacatgcagaacgccgaagagtggttcaagagccgcttcaccgtgctaaccgagagcgccgccaagaacaccgacgctgtgcgcgctgccaaggacgaggtgtcggaaagccgccgcctgctcaaggctaagaccctggagatcgaagcctgccggggtatgaacgaagctctggagaagcagctgcaggagctagaggacaagcagaatgcagacattagcgccatgcaggacacaatcaacaaactggagaatgagctgagaagcacgaagagcgagatggccaggtacctgaaggagtaccaggacctcctcaatgtcaagatggccttggacatcgagattgcagcttacagaaaactcttggaaggcgaagagaccaggctcagtttcaccagcgtgggtagcataaccagcggctactctcagagctcgcaggtcttcggccgttctgcttacagtggcttgcagagcagctcctacttgatgtctgctcgctctttcccagcctactataccagccacgtccaggaagagcagacagaggtcgaggagaccattgaggctacgaaagctgaggaggccaaggatgagcccccctctgaaggagaagcagaagaggaggagaaggagaaagaggagggagaggaagaggaaggcgctgaggaggaagaagctgccaaggatgagtctgaagacacaaaagaagaagaagaaggtggtgagggtgaggaggaagacaccaaagaatctgaagaggaagagaagaaagaggagagtgctggagaggagcaggtggctaagaagaaagattga

SEQ ID NO: 4 - protein, 543 aa, Human
MSSFGYDPYFSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEIAFLKKVHEEEIAELQAQIQYAQISVEMDVSSKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRSTKSEMARYLKEYQDLLNVKMALDIEIAAYRKLLEGEETRLSFTSVGSITSGYSQSSQVFGRSAYSGLQSSSYLMSARSFPAYYTSHVQEEQTEVEETIEATKAEEAKDEPPSEGEAEEEEKEKEEGEEEEGAEEEEAAKDESEDTKEEEEGGEGEEEDTKESEEEEKKEESAGEEQVAKKKD